(19) United States
(12) Patent Application Publication
(10) Pub. No.: US 2017/0145370 A1
(43) Pub. Date: May 25, 2017

(54) CELL CULTURE CONTAINER, CELL IMAGING METHOD, AND CELL CULTURE SYSTEM

(71) Applicant: Panasonic Intellectual Property Management Co., Ltd., Osaka (JP)

(72) Inventors: YUMIKO KATO, Osaka (JP); TAICHI SATO, Kyoto (JP); MASAHIRO IWASAKI, Nara (JP)

(21) Appl. No.: 15/343,432

(22) Filed: Nov. 4, 2016

(30) Foreign Application Priority Data
Nov. 25, 2015 (JP) 2015-229954

(51) Int. Cl. C12M 1/00; C12M 1/34

(52) U.S. Cl. CPC C12M 31/00; C12M 41/46; C12M 41/44

(57) ABSTRACT

A cell culture container includes a container that houses therein a liquid mixture including one or more cells and a culture solution, an irradiator that irradiates the liquid mixture with light, and an image sensor that receives transmitted light that is the light that has been emitted from the irradiator and has passed through the liquid mixture. The light emitted from the irradiator includes a plurality of rays, and the plurality of rays do not cross each other between the irradiator and the image sensor.

### BACKGROUND

1. Technical Field

The present disclosure relates to a cell culture container capable of imaging cells cultured in a culture solution, a cell imaging method using the same, and a cell culture system.

2. Description of the Related Art

Observation of cultured cells is essential in chemical substance screening and clinical examinations, for example. Japanese Unexamined Patent Application Publication (Translation of PCT Application) No. 2009-509543 discloses a cell culture container including an image sensor disposed at the bottom portion thereof and valves for changing a cell staining solution. Cells are irradiated with light from outside the sealed cell culture container, and the light that has passed through the cells is received by the imaging sensor. In this way, the cells are imaged. Since most cells and most culture solutions are colorless, in Japanese Unexamined Patent Application Publication (Translation of PCT Application) No. 2009-509543 cells are stained prior to imaging. Thus, Japanese Unexamined Patent Application Publication (Translation of PCT Application) No. 2009-509543 discloses the valves for a cell staining solution. In addition, Japanese Unexamined Patent Application Publication (Translation of PCT Application) No. 2013-542468 discloses a technique of constructing a high-resolution image from sub-pixel shifted projection images in a scanning projective lensless microscope using incoherent light.

Staining is not an option when cells are continuously cultured and observed. Since most cells are colorless and cultured cells are in a medium (e.g., culture solution), the contrast between the subject and the background is very low, making it difficult to image the cells. Further, continuous observation of the cultured cells is desirably performed without removing the cultured cells from an incubator.

### SUMMARY

One non-limiting and exemplary embodiment provides a cell culture container, a cell imaging method, and a cell culture system capable of imaging cultured cells in an incubator without staining the cells.

In one general aspect, the techniques disclosed here feature a cell culture container including a container that houses therein a liquid mixture including one or more cells and a culture solution, an irradiator that irradiates the liquid mixture with light, and an image sensor that receives transmitted light. The transmitted light is the light that has been emitted from the irradiator and has passed through the liquid mixture. The light emitted from the irradiator includes a plurality of rays, and the plurality of rays do not cross each other between the irradiator and the image sensor. The container has a mark on a side portion thereof, the side portion is located between a top portion of the container and a bottom portion of the container, and a surface of the liquid mixture is located between the top portion and the bottom portion. The irradiator includes an emission surface from which the light is emitted, and the emission surface is located below the surface of the liquid mixture if the container is filled with the liquid mixture up to a height indicated by the mark.

According to the aspect of the present disclosure, cultured cells are successfully imaged in an incubator without staining the cells.

It should be noted that general or specific embodiments may be implemented as an apparatus, a method, or a system, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a perspective view of a dish-type cell culture container according to a first embodiment;

FIG. 1B is a perspective view of a flask-type cell culture container according to the first embodiment;

FIG. 2A is a cross-sectional view of the dish-type cell culture container schematically illustrating an exemplary usage of the dish-type cell culture container according to the first embodiment;

FIG. 2B is a cross-sectional view of the flask-type cell culture container schematically illustrating an exemplary usage of the flask-type cell culture container according to the first embodiment;

FIG. 3 is a schematic diagram illustrating an example of a detailed structure of an irradiator according to the first embodiment;

FIG. 4A is a schematic diagram illustrating another example of the detailed structure of the irradiator according to the first embodiment;

FIG. 4B is a schematic diagram illustrating another example of the detailed structure of the irradiator according to the first embodiment;

FIG. 5 is a schematic diagram illustrating another example of the detailed structure of the irradiator according to the first embodiment;

FIG. 6A is a cross-sectional view of a dish-type cell culture container according to a first modification of the first embodiment;

FIG. 6B is a cross-sectional view of a flask-type cell culture container according to the first modification of the first embodiment;

FIG. 7 is a flowchart illustrating a cell imaging method according to the first modification of the first embodiment;

FIG. 8A is a diagram for describing advantageous effects of the first modification of the first embodiment;

FIG. 8B is a diagram for describing advantageous effects of the first modification of the first embodiment;

FIG. 8C is a diagram for describing advantageous effects of the first modification of the first embodiment;

FIG. 9A is a cross-sectional view of a dish-type cell culture container according to a second modification of the first embodiment;

FIG. 9B is a cross-sectional view of a flask-type cell culture container according to the second modification of the first embodiment;

FIG. 10A is a perspective view of a dish-type cell culture container according to a third modification of the first embodiment;

FIG. 10B is a perspective view of a flask-type cell culture container according to the third modification of the first embodiment;

FIG. 11 is a cross-sectional view illustrating an example of a cell culture container according to a second embodiment;

FIG. 12 is a cross-sectional view illustrating another example of a cell culture container according to the second embodiment;

FIG. 13 is a perspective view of a dish-type cell culture container according to a third embodiment;

FIG. 14 is a cross-sectional view of the cell culture container when a main body is covered with a lid in the third embodiment;

FIG. 15 is a block diagram illustrating a functional configuration of the cell culture container according to the third embodiment;

FIG. 16 is a flowchart illustrating an example of an operation performed in the cell culture container according to the third embodiment;

FIG. 17 is a block diagram illustrating another example of the functional configuration of the cell culture container according to the third embodiment;

FIG. 18 is a block diagram illustrating a functional configuration of a cell culture container according to a first modification of the third embodiment;

FIG. 19 is a perspective view of a dish-type cell culture container and a tray according to a fourth embodiment;

FIG. 20 is a perspective view of a dish-type cell culture container and a tray according to a first modification of the fourth embodiment;

FIG. 21 is a block diagram illustrating a functional configuration of the cell culture container and the tray according to the first modification of the fourth embodiment;

FIG. 22 is a functional block diagram of a cell culture system according to a fifth embodiment;

FIG. 23A is a diagram schematically illustrating an operation performed by the cell culture system according to the fifth embodiment;

FIG. 23B is a diagram schematically illustrating the operation performed by the cell culture system according to the fifth embodiment;

FIG. 23C is a diagram schematically illustrating the operation performed by the cell culture system according to the fifth embodiment;

FIG. 23D is a diagram schematically illustrating the operation performed by the cell culture system according to the fifth embodiment;

FIG. 23E is a diagram schematically illustrating the operation performed by the cell culture system according to the fifth embodiment;

FIG. 24 is a flowchart illustrating the operation performed by the cell culture system according to the fifth embodiment;

FIG. 25 is a functional block diagram of a cell culture system according to a first modification of the fifth embodiment; and

FIG. 26 is a functional block diagram of a cell culture system according to a second modification of the fifth embodiment.

### DETAILED DESCRIPTION

A cell culture container according to an aspect of the present disclosure includes a container that houses therein a liquid mixture including one or more cells and a culture solution, an irradiator that irradiates the liquid mixture with light, and an image sensor that receives transmitted light. The transmitted light is the light that has been emitted from the irradiator and has passed through the liquid mixture. The light emitted from the irradiator includes a plurality of rays, and the plurality of rays do not cross each other between the irradiator and the image sensor. The container has a mark on a side portion thereof, the side portion is located between a top portion of the container and a bottom portion of the container, and a surface of the liquid mixture is located between the top portion and the bottom portion. The irradiator includes an emission surface from which the light is emitted, and the emission surface is located below the surface of the liquid mixture if the container is filled with the liquid mixture up to a height indicated by the mark.

With this configuration, the liquid mixture can be irradiated with light including rays that do not cross each other between the irradiator and the image sensor. Thus, an optical image representing the shape and dimensions of the cells is accurately formed on a light-receiving surface of the image sensor, and consequently the cultured cells are successfully imaged in an incubator without staining the cells. In addition, with this configuration, the emission surface from which the light is emitted is successfully located below the surface of the liquid mixture when the container is filled with the liquid mixture up to the height indicated by the mark. Thus, refraction of light at the surface of the liquid mixture is successfully avoided, and consequently the shape and dimensions of the cells are successfully derived more easily from the obtained image.

The irradiator may be disposed to protrude from the container toward inside the container.

With this configuration, the irradiator can be disposed to protrude from the container toward inside the container. Thus, the irradiator is successfully placed closer to the liquid mixture in the container, and consequently an optical image that represents the shape and dimensions of the cells more accurately is successfully formed on the light-receiving surface of the image sensor.

The plurality of rays may be parallel to each other between the irradiator and the image sensor.

With this configuration, the liquid mixture can be irradiated with light (parallel light) including rays that are parallel to each other between the irradiator and the image sensor. Consequently, an optical image representing the shape and dimensions of the cells is successfully formed on the light-receiving surface of the image sensor accurately.

The irradiator may include a limiting filter that limits a traveling direction of the light, and the light emitted from the irradiator may be light that has passed through the limiting filter.

With this configuration, the irradiator is able to easily irradiate the liquid mixture with parallel light by using the limiting filter that limits the traveling direction of the light.

The irradiator may include a collimating lens, and the light emitted from the irradiator may be light that has passed through the collimating lens.

With this configuration, the irradiator is able to easily irradiate the liquid mixture with parallel light by using the collimating lens.

The plurality of rays may be diffused. The irradiator may include a pinhole, and the light emitted from the irradiator may be light that has passed through the pinhole.

With this configuration, the liquid mixture can be irradiated with diffused light that has passed through the pinhole. Consequently, an optical image representing the shape and dimensions of the cells is successfully formed on the light-receiving surface of the image sensor accurately. In addition, the irradiator is able to easily irradiate the liquid mixture with diffused light by using the pinhole.

The side portion may have a light-shielding property.

With this configuration, the side portion of the container can have a light-shielding property. Thus, an amount of ambient light that passes through the side portion of the container and is incident onto the container from outside the container is successfully reduced, and consequently an optical image representing the shape and dimensions of the cells more accurately is successfully formed on the light-receiving surface of the image sensor.

The bottom portion of the container may have a region not having the image sensor therein, and the region may have a light-shielding property.

With this configuration, a region of the bottom portion of the container not having the image sensor therein can have a light-shielding property. Accordingly, an amount of ambient light that passes through the bottom portion of the container and is incident onto the container from outside the container is successfully reduced, and consequently an optical image representing the shape and dimensions of the cells more accurately can be formed on the light-receiving surface of the image sensor.

A cell imaging method according to an aspect of the present disclosure includes placing an emission surface of an irradiator below a surface of a liquid mixture held in a container, the liquid mixture including one or more cells and a culture solution, the irradiator being configured to emit, from the emission surface, light including a plurality of rays not crossing each other between the irradiator and an image sensor; irradiating, by the irradiator, the liquid mixture with the light; and receiving, by the image sensor, transmitted light, wherein the transmitted light is the light that has been emitted from the irradiator and has passed through the liquid mixture, wherein the container is filled with the liquid mixture up to a height indicated by a mark, and wherein the mark is on a side portion of the container, the side portion is located between a top portion of the container and a bottom portion of the container, and the surface of the liquid mixture is located between the top portion and the bottom portion.

With this configuration, the emission surface from which the light is emitted is successfully located below the surface of the liquid mixture when the container is filled with the liquid mixture up to the height indicated by the mark. Thus, refraction of light at the surface of the liquid mixture is successfully avoided, and consequently the shape and dimensions of the cells are successfully derived more easily from the obtained image.

A cell culture system according to an aspect of the present disclosure includes a container that stores a liquid mixture including one or more cells and a culture solution, an irradiator that irradiates the liquid mixture with light, an image sensor that images the cells in the container, the image sensor being disposed at a bottom portion of the container, a sensor that detects whether a predetermined amount of liquid mixture is stored in the container, and a controller that controls the image sensor. In a case where the sensor detects that the predetermined amount of liquid mixture is stored in the container, the controller controls the image sensor so as to image the cells in the container. In a case where the sensor does not detect that the predetermined amount of liquid mixture is stored in the container, the controller controls the image sensor so as not to image the cells in the container.

The controller may further output an alarm indicating that the predetermined amount of liquid mixture is not stored in the container in the case where the sensor does not detect that the predetermined amount of liquid mixture is stored in the container.

A cell culture container according to another aspect of the present disclosure includes a container that houses therein a liquid mixture including one or more cells and a culture solution, an irradiator that irradiates the liquid mixture with light including first rays that do not cross each other, and an image sensor that receives resultant light output from the liquid mixture, no condenser lens being provided between the liquid mixture and the image sensor, the resultant light corresponding to the light. A mark is on a side of the container, and the side is located between a top of the container and a bottom of the container. An emission surface of the irradiator from which the light is emitted is located between the bottom and a level indicated by the mark.

The first rays may be third rays while passing through the liquid mixture, the resultant light may include second rays, and, with the container housing the liquid mixture from the bottom to the level, the second rays may not cross each other and the third lays may not cross each other.

A cell culture container according to embodiments will be specifically described below with reference to the accompanying drawings.

Each of the embodiments described below provides a general or specific example. The values, shapes, components, arranged positions and connections of the components, steps, orders of the steps, etc., given in the following embodiments are merely illustrative, and are not intended to limit the claims. In addition, among the components in the following embodiments, a component not recited in any of the independent claims indicating the most generic concept is described as an optional component.

### First Embodiment

A cell culture container according to a first embodiment will be described. The cell culture container may be a dish-type container called petri dish or a flask-type container that is horizontally placed.

### Structure of Cell Culture Container

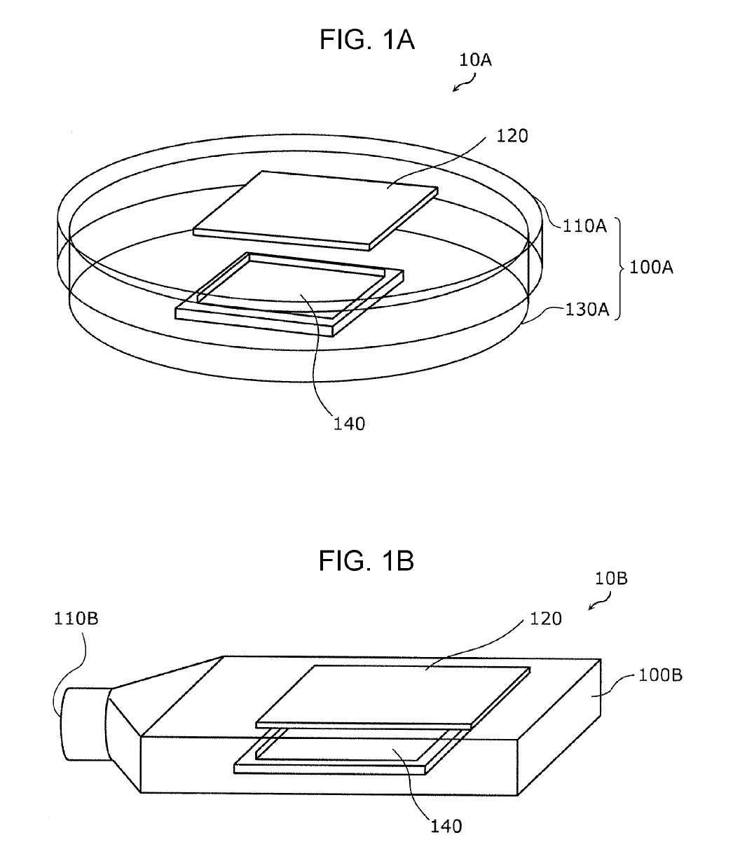

FIG. 1A is a perspective view of a dish-type cell culture container 10A (also simply referred to as the cell culture container 10A) according to the first embodiment. Referring to FIG. 1A, the cell culture container 10A includes a container unit 100A, an irradiator 120, and an image sensor 140.

The container unit 100A is a container that holds a liquid mixture including one or more cells and a culture solution. That is, the container unit 100A is a container that houses the liquid mixture therein. The container unit 100A is a transparent container composed of glass or a resin, for example. The container unit 100A includes a lid 110A and a main body 130A.

The main body 130A is a bottomed cylindrical member that constitutes a bottom portion and a side portion of the container unit 100A.

The lid 110A is a bottomed cylindrical member that covers an opening of the main body 130A when it is fitted to the main body 130A. The lid 110A constitutes a top portion of the container unit 100A.

The irradiator 120 is disposed on an inner surface of the lid 110A. The irradiator 120 irradiates a liquid mixture held in the container unit 100A with light. The light passes through the liquid mixture and exits from the liquid mixture as transmitted light. The transmitted light is light that has been emitted from the irradiator 120 and has passed through the liquid mixture, that is, light that has been refracted and attenuated by a liquid mixture that is a translucent substance. Specifically, the irradiator 120 is fixed to the inner surface of the lid 110A and irradiates the liquid mixture held in the container unit 100A with non-crossing light from above. Note that the irradiator 120 may be fixed onto an outer surface of the lid 110A.

The term “non-crossing light” refers to light that is incident on each pixel of the image sensor 140 from a single direction. That is, a plurality of rays of light emitted from the irradiator 120 do not cross each other between the irradiator 120 and the image sensor 140. For example, the non-crossing light is diffused light from a point light source or parallel light.

The image sensor 140 is disposed at the bottom portion of the container unit 100A and receives the transmitted light that has exited from the liquid mixture. The image sensor 140 is a solid-state imaging element, for example, a charge coupled device (CCD) image sensor or a complementary metal oxide semiconductor (CMOS) image sensor. The image sensor 140 includes a plurality of pixels arranged in a matrix. The non-crossing light emitted from the irradiator 120 is incident on each of the pixels of the image sensor 140 from a single direction. The image sensor 140 obtains an optical image of cells that is formed on the light-receiving surface thereof as a result of irradiation of the cells with the non-crossing light.

Note that the cell culture container is not limited to the dish-type cell culture container 10A illustrated in FIG. 1A and may be a flask-type cell culture container 10B illustrated in FIG. 1B.

FIG. 1B is a perspective view of the flask-type cell culture container 10B (also simply referred to as the cell culture container 10B) according to the first embodiment. The cell culture container 10B includes a container unit 100B, the irradiator 120, and the image sensor 140.

The container unit 100B is a container that holds a liquid mixture including one or more cells and a culture solution. That is, the container unit 100B is a container that houses the liquid mixture therein. The container unit 100B is composed of glass or a resin, for example. The container unit 100B has an opening at a side portion thereof, and the opening is covered with a cap 110B.

The irradiator 120 is disposed at a top portion of the container unit 100B. The irradiator 120 irradiates a liquid mixture held in the container unit 100B with light. The light passes through the liquid mixture and exits from the liquid mixture as transmitted light. Specifically, the irradiator 120 is fixed onto an outer top surface of the container unit 100B and irradiates the liquid mixture held in the container unit 100B with non-crossing light. Note that the irradiator 120 may be fixed onto an inner top surface of the container unit 100B.

The image sensor 140 is disposed at the bottom surface of the container unit 100B. The image sensor 140 obtains an optical image of cells that is formed on the light-receiving surface thereof as a result of irradiation of the cells with the non-crossing light.

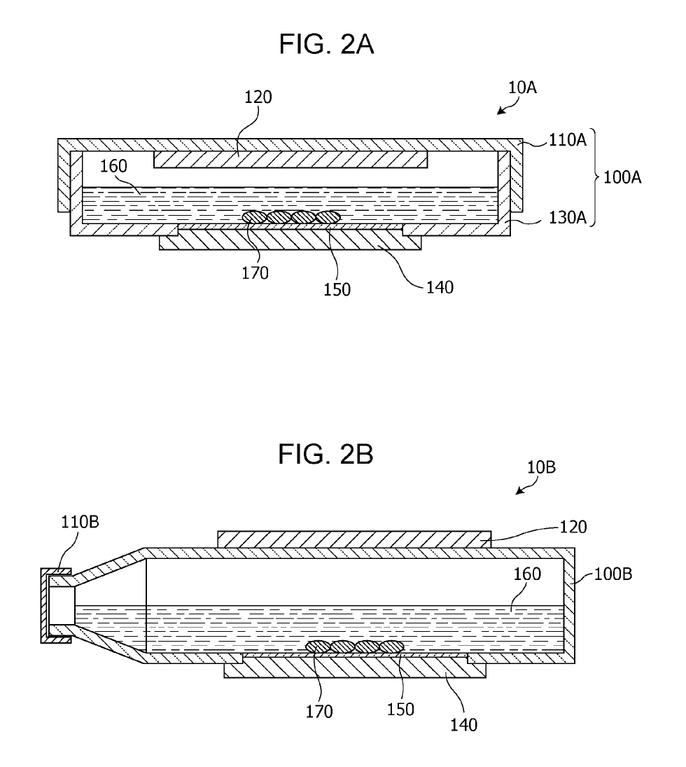

FIG. 2A is a cross-sectional view of the dish-type cell culture container 10A according to the first embodiment. FIG. 2B is a cross-sectional view of the flask-type cell culture container 10B according to the first embodiment. Referring to FIGS. 2A and 2B, cells 170 and a culture solution 160 are held in the container units 100A and 100B, respectively.

As illustrated in FIGS. 2A and 2B, the image sensor 140 is disposed at the bottom portion of the container units 100A and 100B, respectively. Specifically, the image sensor 140 is fitted to an opening formed at the bottom portion of the container units 100A and 100B. The light-receiving surface of the image sensor 140 is covered with a transparent protection film 150 and is exposed to a space inside the container units 100A and 100B. The cells 170 are immersed in the culture solution 160 that fills the container units 100A and 100B and are cultured in a state where the cells 170 are in direct contact with the transparent protection film 150. In FIGS. 2A and 2B, there is no condenser lens between the cells 170 and the image sensor 140.

Although the cells 170 are cultured in a state where the cells 170 are in direct contact with the transparent protection film 150 covering the image sensor 140 in FIGS. 2A and 2B, a thin transparent glass having a thickness of 0.1 mm or less (e.g., a cover slip used for microscopic observation) may be disposed between the cells 170 and the transparent protection film 150.

When the cells 170 are cultured in a state where the cells 170 are in direct contact with the transparent protection film 150 or the thin transparent glass, a material such as an extracellular matrix that promotes fixing of the cells 170 may be applied onto the transparent protection film 150 or the thin transparent glass. When the cells 170 are cells that do not fix, such as early embryos, the culture solution 160 may be placed on the transparent protection film 150 or the thin transparent glass so that the cells 170 are immersed in the culture solution 160.

As described above, the cells 170 that are the subject of imaging are cultured in a state where the cells 170 are placed on the light-receiving surface of the image sensor 140 with a set of the transparent protection film 150 and the thin transparent glass or the transparent protection film 150 alone interposed therebetween.

The irradiator 120 is disposed at the top portion of the container units 100A and 100B. Specifically, in FIG. 2A, the irradiator 120 is disposed on the inner surface of the lid 110A to be located above the image sensor 140, whereas in FIG. 2B, the irradiator 120 is disposed on the outer top surface of the container unit 100B to be located above the image sensor 140. The irradiator 120 irradiates the liquid mixture including the cells 170 and the culture solution 160 with non-crossing light from above.

### Structure of Irradiator

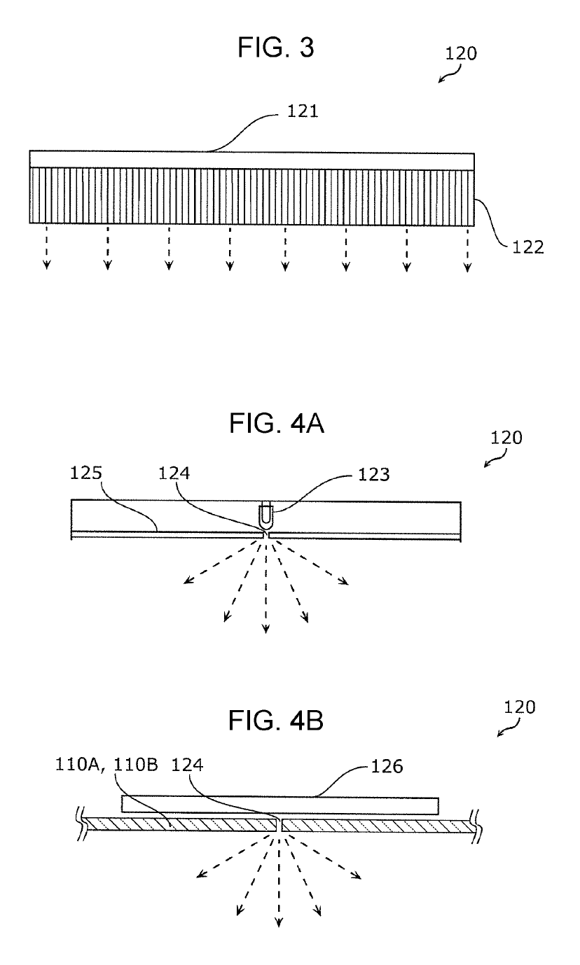

FIG. 3 is a schematic diagram illustrating an example of a detailed structure of the irradiator 120 according to the first embodiment. Referring to FIG. 3, the irradiator 120 emits parallel light as the non-crossing light. That is, a plurality of rays representing the light emitted by the irradiator 120 are parallel to each other between the irradiator 120 and the image sensor 140. The irradiator 120 includes an area light source 121 and a limiting filter 122.

The area light source 121 may be implemented by a light source that emits light from the entire surface thereof, such as an organic electroluminescence illumination, for example, or may be implemented using a light guiding panel.

The limiting filter 122 is a filter that limits the traveling direction (angle) of light by using liquid crystal or the like. The limiting filter 122 allows only light that travels in a direction perpendicular to the light-receiving surface of the image sensor 140 disposed at the bottom portion of the cell culture containers 10A and 10B to pass therethrough. Consequently, the irradiator 120 is able to emit parallel light that is perpendicular to the light-receiving surface of the image sensor 140. That is, the irradiator 120 emits light that has passed through the limiting filter 122.

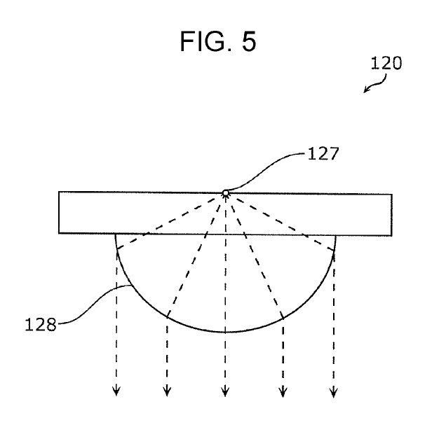

Each of FIGS. 4A and 4B is a schematic diagram illustrating another example of the detailed structure of the irradiator 120 according to the first embodiment. The irradiator 120 serves as a point light source.

Referring to FIG. 4A, the irradiator 120 includes, for example, a light source 123 and a light-shielding plate 125 having a pinhole 124.

The light source 123 is disposed, for example, at the top portion of the container units 100A and 100B and evenly emits light in multiple directions. The light-shielding plate 125 is disposed to be located below the light source 123 and be parallel to the upper inner surface of the container units 100A and 100B such that the light-shielding plate 125 covers the entire top surface of the container units 100A and 100B where the light source 123 is disposed. The light-shielding plate 125 has the pinhole 124 which is an extremely small hole that allows light to passes therethrough. The pinhole 124 is located right above the image sensor 140.

Referring to FIG. 4B, a light source 126 (e.g., area light source) that emits light in a random direction is disposed outside the container units 100A and 100B. The top portion of the container units 100A and 100B has a light-shielding property. Further, the top portion of the container units 100A and 100B has the pinhole 124.

The pinhole 124 is located right above the image sensor 140. The pinhole 124 allows light emitted from the light source 126 in a random direction to pass therethrough to emit non-crossing light that is diffused in multiple directions. That is, light emitted from the light source 126 passes through the pinhole 124 and is diffused. The pinhole 124 has a diameter of 0.1 mm, for example.

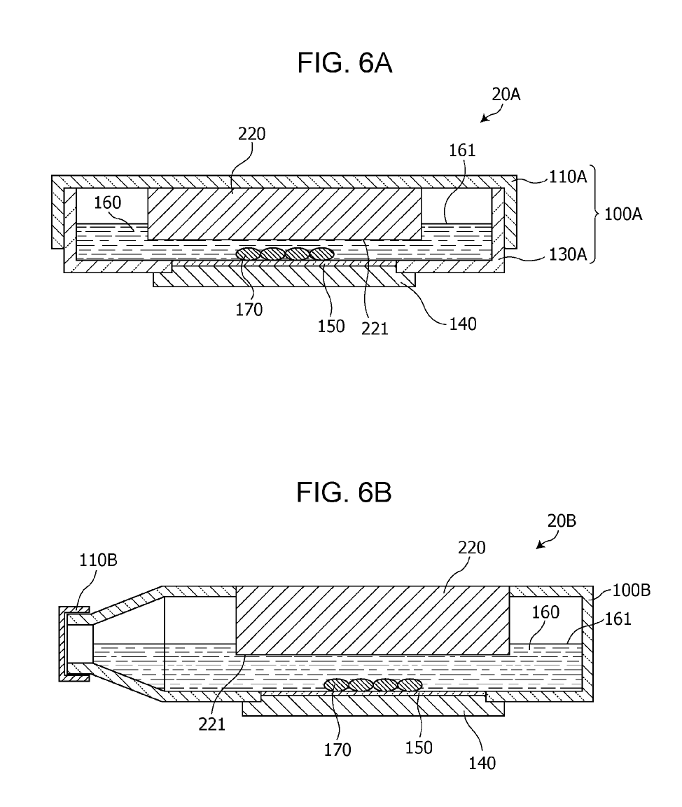

FIG. 5 is a schematic diagram illustrating another example of the detailed structure of the irradiator 120 according to the first embodiment.

Referring to FIG. 5, the irradiator 120 includes a point light source 127 and a lens 128.

The point light source 127 is disposed at the focal position of the lens 128. Thus, the entire surface of the lens 128 on the point light source 127 side is irradiated with light emitted from the point light source 127. The point light source 127 is implemented by using a light-emitting diode (LED) illumination and a pinhole, for example. The lens 128 is a collimating lens, for example. The light emitted from the lens 128 is parallel light perpendicular to the light-receiving surface of the image sensor 140. That is, the irradiator 120 emits parallel light through the lens 128.

With the configuration illustrated in any of FIGS. 3 to 5, the irradiator 120 emits parallel light or diffused light using the point light source toward the light-receiving surface of the image sensor 140 disposed at the bottom portion of the container units 100A and 100B.

The light emitted by such an irradiator 120 reaches the light-receiving surface of the image sensor 140 after passing through the cells 170 located on the light-receiving surface of the image sensor 140. Since the light emitted by the irradiator 120 at that time is parallel light or diffused light from the point light source (i.e., non-crossing light), the light is incident on each pixel of the image sensor 140 from a single direction. Part of the light is absorbed by the cells 170 when the light passes through the cells 170, and consequently an optical image of the cells 170 is formed on the light-receiving surface of the image sensor 140. Thus, the strength of the light that reaches pixels corresponding to the optical image decreases. The image sensor 140 successfully images the cells 170 by obtaining an optical image of the cells 170 formed on the light-receiving surface by the non-crossing light.

### Advantageous Effects

As described above, in the cell culture containers 10A and 10B according to the first embodiment, the irradiator 120 that emits non-crossing light toward inside the cell culture containers 10A and 10B can be disposed at the top portion of the container units 100A and 100B, respectively, and the image sensor 140 can be disposed at the bottom portion of the container units 100A and 100B. Since this configuration enables an optical image representing the shape and dimensions of the cells 170 to be accurately formed on the light-receiving surface of the image sensor 140, the cultured cells 170 are successfully imaged in an incubator without being stained.

In addition, in the cell culture containers 10A and 10B according to the first embodiment, parallel light can be used as non-crossing light. Thus, an optical image representing the shape and dimensions of the cells 170 is successfully formed on the light-receiving surface of the image sensor 140 accurately. In addition, the irradiator 120 is able to easily emit parallel light by using a limiting filter that limits the traveling direction of light or a collimating lens.

Further, in the cell culture containers 10A and 10B according to the first embodiment, diffused light emitted from a point light source can be used as non-crossing light. Thus, an optical image representing the shape and dimensions of the cells 170 is successfully formed on the light-receiving surface of the image sensor 140 accurately. In addition, the irradiator 120 is able to easily emit diffused light through a pinhole.

### First Modification of First Embodiment

A first modification of the first embodiment will be described next. In the first embodiment, the irradiator 120 is disposed at the top portion of the container units 100A and 100B of the cell culture containers 10A and 10B to be apart from the surface of the liquid mixture. In contrast, in the first modification, a light emission surface of an irradiator is located at a position lower than the surface of the liquid mixture held in the cell culture container, that is, a position closer to the light-receiving surface of the image sensor 140 than to the surface of the liquid mixture. That is, the light emission surface of the irradiator is immersed in the liquid mixture. The first modification will be described below by focusing on differences from the first embodiment.

### Structure of Cell Culture Container

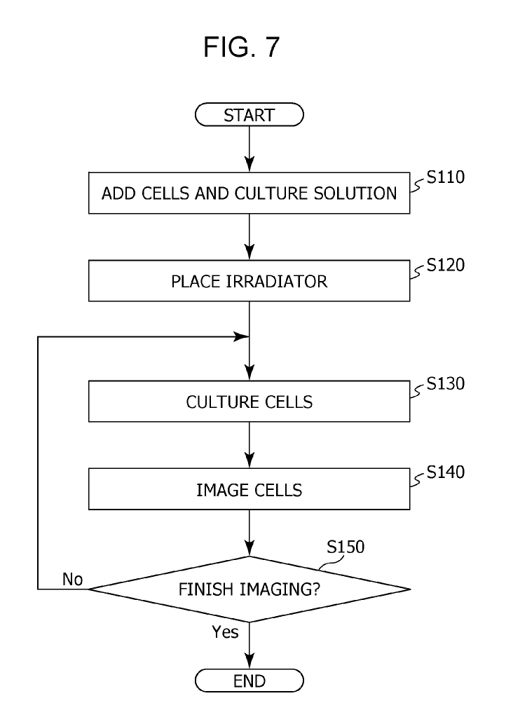

FIG. 6A is a cross-sectional view of a dish-type cell culture container 20A according to the first modification of the first embodiment. FIG. 6B is a cross-sectional view of a flask-type cell culture container 20B according to the first modification of the first embodiment. In FIGS. 6A and 6B, components that are substantially the same as those illustrated in FIGS. 2A and 2B are denoted by the same reference signs, and a detailed description thereof is omitted.

Referring to FIG. 6A, the dish-type cell culture container 20A (also simply referred to as the cell culture container 20A) includes the container unit 100A, an irradiator 220, and the image sensor 140.

The irradiator 220 protrudes from the container unit 100A toward inside the container unit 100A. In the first modification, the irradiator 220 is disposed on a top portion of the container unit 100A and emits non-crossing light (parallel light or diffused light from a point light source). Specifically, the irradiator 220 protrudes from the top portion of the container unit 100A toward inside the container unit 100A. A non-crossing light emission surface 221 of the irradiator 220 is located in the liquid mixture including the cells 170 and the culture solution 160 held in the container unit 100A. That is, the non-crossing light emission surface 221 of the irradiator 220 is located below a surface 161 of the liquid mixture and above the bottom portion of the container unit 100A.

Referring to FIG. 6B, the flask-type cell culture container 20B (also simply referred to as the cell culture container 20B) includes the container unit 100B, the irradiator 220, and the image sensor 140. The irradiator 220 protrudes from the top portion of the container unit 100B toward inside the container unit 100B. The non-crossing light emission surface 221 of the irradiator 220 is located in the liquid mixture held in the container unit 100B.

The surface of the irradiator 220 is covered with a transparent material that does not refract light to cope with adhesion of the liquid mixture.

The irradiator 220 may emit parallel light as illustrated in FIG. 3 or diffused light as illustrated in FIG. 4A. When the irradiator 220 is configured as illustrated in FIG. 4A, the pinhole 124 is filled with a transparent material that does not refract light, for example, a fluorine-containing semi-aromatic polyimide. Thus, the liquid mixture does not go above the light-shielding plate 125 through the pinhole 124. In addition, the light-shielding plate 125 is composed of a material that is not degraded by the liquid mixture.

### Cell Imaging Method

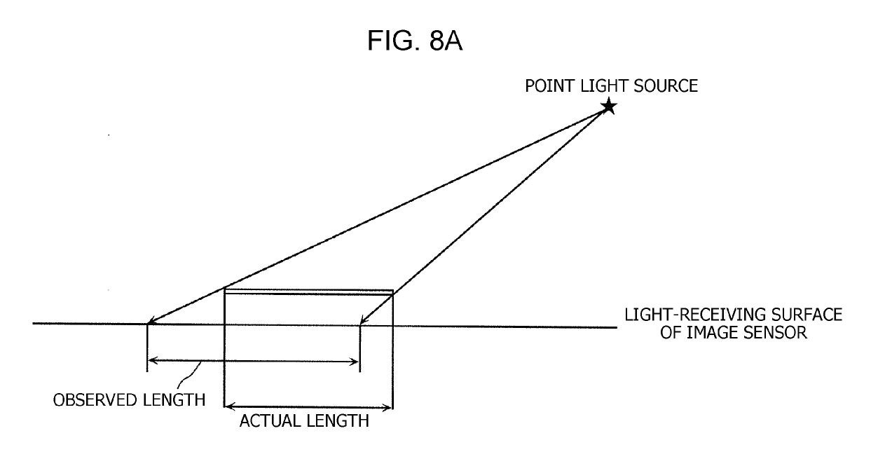

A method for imaging the cells 170 by using the cell culture container 20A or 20B configured in the above manner will be described next. FIG. 7 is a flowchart illustrating a cell imaging method according to the first modification of the first embodiment.

First, the liquid mixture including the cells 170 and the culture solution 160 is held in the container unit 100A or 100B (S110). Then, the non-crossing light emission surface 221 of the irradiator 220 is placed in the liquid mixture (S120). That is, the non-crossing light emission surface 221 is placed below the surface of the liquid mixture. For example, the lid 110A of the cell culture container 20A is fitted to the main body 130A, and consequently the non-crossing light emission surface 221 of the irradiator 220 is placed below the surface of the liquid mixture. In addition, for example, the cell culture container 20B is placed horizontally as illustrated in FIG. 6B, and consequently the non-crossing light emission surface 221 of the irradiator 220 is placed below the surface of the liquid mixture.

Subsequently, the cells 170 held in the cell culture container 20A is cultured in an incubator (S130). The irradiator 220 irradiates the liquid mixture with light, and the image sensor 140 receives the light that has passed through the liquid mixture (S140).

Then, it is determined whether to finish imaging (S150). If it is determined to finish imaging (Yes in S150), the process ends. If it is not determined to finish imaging (No in S150), the process returns to step S130.

### Advantageous Effects

As described above, in the cell culture containers 20A and 20B according to the first modification, the irradiator 220 can be disposed to protrude from the top portion of the container units 100A and 100B toward inside the container units 100A and 100B. Thus, the irradiator 220 can be brought closer to the cells 170 which are the subject, and consequently an optical image representing the shape and dimensions of the cells 170 more accurately is successfully formed on the light-receiving surface of the image sensor 140.

In addition, in the cell culture containers 20A and 20B according to the first modification, the non-crossing light emission surface 221 of the irradiator 220 can be placed in the liquid mixture. Thus, refraction of light at the surface 161 of the liquid mixture is successfully avoided, and consequently the shape and dimensions of the cells 170 is successfully derived more easily from the obtained image.

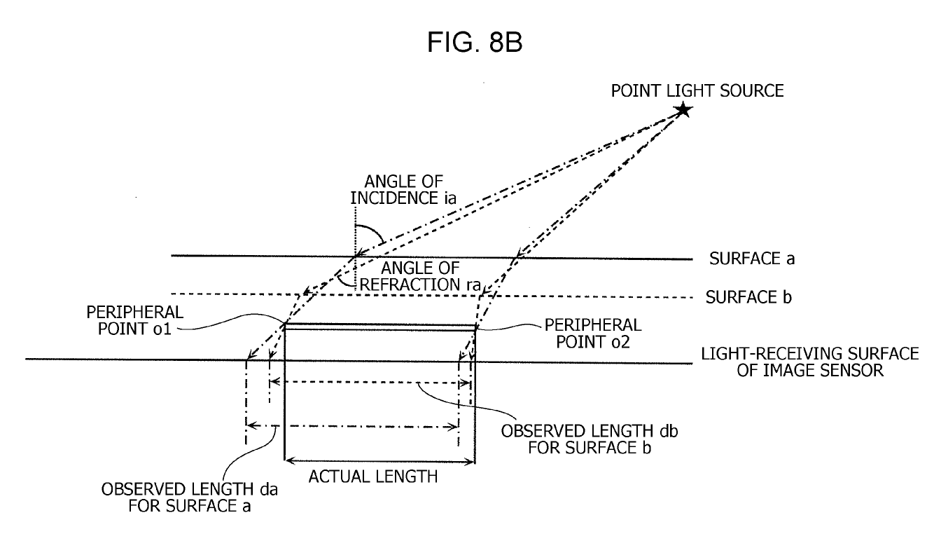

Now, such advantageous effects of the first modification will be specifically described with reference to FIGS. 8A to 8C. FIGS. 8A to 8C are diagrams for describing the advantageous effects of the first modification of the first embodiment. The case where the irradiator 220 emits diffused light will be described here.

FIG. 8A is a diagram for describing an optical image of a cell formed on a light-receiving surface of an image sensor by using diffused light emitted from a point light source. FIG. 8B is a diagram for describing an influence of refraction of light at the surface of the liquid mixture. FIG. 8C is a diagram for describing a relationship between the length of the optical image of the subject observed in the first modification of the first embodiment and the actual length of the subject.

As the distance from the position right under the light source becomes larger, the diffused light emitted from the point light source diffuses by a larger degree. Thus, an optical image of the subject located between the point light source and the image sensor (i.e., the shape of an optical image of the subject formed on the light-receiving surface of the image sensor) changes in accordance with the distance from the point light source. FIG. 8A indicates that the length of the optical image of the subject formed on the light-receiving surface of the image sensor is greater than the actual length of the subject.

The cell culture container holds the culture solution therein, and the cells are cultured in the culture solution. When imaging is performed by using the image sensor disposed at the bottom portion of the container unit and the irradiator disposed at the top portion of the container unit, the surface of the liquid mixture is located between the subject and the irradiator. The height from the image sensor disposed at the bottom portion of the cell culture container to the surface of the liquid mixture changes in accordance with an amount of liquid mixture held in the cell culture container. Since light refracts at the surface of the liquid mixture, the length of the optical image of the subject formed on the light-receiving surface of the image sensor is influenced not only by diffusion of light emitted from the point light source but also by refraction of the light at the surface of the liquid mixture.

FIG. 8B indicates that the length of the optical image of the subject formed on the light-receiving surface of the image sensor changes depending on the height from the image sensor to the surface of the liquid mixture. When the surface of the liquid mixture is at the height of the surface a denoted by a solid line, rays emitted from the point light source and denoted by a dot-dash line refract at the surface a of the liquid mixture, travel in straight lines in the liquid mixture, and are blocked by the subject, whereby an optical image of the subject is formed on the light-receiving surface of the image sensor. The length of the optical image formed on the light-receiving surface of the image sensor is da.

In contrast, when the surface of the liquid mixture is at the height of the surface b, rays emitted from the point light source and denoted by a dash line refract at the surface b of the liquid mixture, travel in straight lines in the liquid mixture, and are blocked by the subject, whereby an optical image of the subject is formed on the light-receiving surface of the image sensor. The length of the optical image of the subject formed on the light-receiving surface of the image sensor is db.

For example, to determine the actual length of the subject from the length da, an angle of incidence at which rays that touch the peripheral points o1 and o2 of the subject and reach the image sensor are incident on the light-receiving surface of the image sensor needs to be determined. However, since the rays refract at the surface a of the liquid mixture, an angle of refraction ra at which the rays that touch the peripheral points o1 and o2 of the subject refract needs to be determined.

A ratio between the angle of incidence ia of the rays that are incident onto the surface a of the liquid mixture from the point light source and the angle of refraction ra at the surface a is equal to a relative index of refraction of the gas and the culture solution held in the container. Thus, the angle of incidence ia is determined based on the height of the surface a and the distance from the position where the ray is incident on the surface a to the position right under the point light source on the plane of the surface a.

That is, to determine the actual length of the subject from the length da, the angle of refraction ra needs to be determined. To determine the angle of refraction ra, the distance from the surface a to the image sensor and the distance from the point light source to the surface a need to be determined. Specifically, to determine the angle of refraction ra, a combination of the angle of incidence ia and the angle of refraction ra that satisfies a condition is extracted based on the distance from the surface a to the image sensor, the distance from the point light source to the surface a, and the relative index of refraction of the gas and the culture solution held in the container.

Since information regarding the height of the surface of the liquid mixture needs to be obtained if the surface of the liquid mixture is located between the subject and the point light source in this manner, it is difficult to determine the angle of refraction ra that determines the angle of incidence ia of rays onto the image sensor. Consequently, it is difficult to determine the actual length of the subject from the obtained image and determine the actual shape of the subject.

In contrast, if the light emission surface from which diffused light from the point light source is emitted is located in the liquid mixture (i.e., if the surface of the liquid mixture does not located between the light emission surface and the subject and between the light emission surface and the image sensor), light travels in straight lines without refraction. In this case, the shape of the subject can be determined from the obtained image even if the height of the surface of the liquid mixture is not used.

FIG. 8C illustrates distances between components. To estimate the actual shape of the subject from the shape of the optical image of the subject formed on the light-receiving surface of the image sensor, the length of the optical image needs to be corrected. For example, the actual length dt of the subject is estimated from the length do of the optical image of the subject formed on the light-receiving surface of the image sensor in the following procedure in FIG. 8C.

Distances from a point right under the point light source on the light-receiving surface of the image sensor to two reaching points p1 and p2 which respective rays that touch the peripheral points o1 and o2 of the subject reach the image sensor are denoted by w1 and w2, respectively. A distance from the light-receiving surface of the image sensor to the point light source is denoted by hl, and a distance from the light-receiving surface of the image sensor to the subject is denoted by hs. Positions to which the peripheral points o1 and o2 of the subject are mapped onto the light-receiving surface of the image sensor are respectively denoted by S1 and S2. Since a triangle created by the vertical line extending from the point light source to the light-receiving surface of the image sensor, the light-receiving surface of the image sensor, and the straight line extending from the point light source to the reaching point p1 or p2 is similar to a triangle created by the vertical line extending from the point light source to the light-receiving surface of the image sensor, the subject surface, and the straight line extending from the point light source to the reaching point p1 or p2, the positions S1 and S2 are denoted as follows:

S1=(p1(hl−hs))/hl

S2=(p2(hl=hs))/hl.

Accordingly, the actual length dt can be determined as follows:

dt=S1−S2=(p1−p2)(hl−hs)/hl=do(hl−hs)/hl.

For example, when the hs and hl are known to be 0.1 mm and 1 mm, respectively, and when the length of the optical image of the subject do=0.5 mm is determined from the obtained image, the actual length dt of the subject is determined to be 0.45 mm (=0.5(1−0.1)/1).

As described above, refraction of light at the surface 161 of the liquid mixture is successfully avoided by placing the non-crossing light emission surface 221 of the irradiator 220 in the liquid mixture. Consequently, the shape and dimensions of the cell is successfully derived easily from the obtained image without requiring detection of the height of the surface 161 of the liquid mixture.

### Second Modification of First Embodiment

A second modification of the first embodiment will be described next. In the second modification, the configuration of the irradiator differs from that of the first modification of the first embodiment. The irradiator of the second modification includes a lens as illustrated in FIG. 5. The second modification will be described below by focusing on differences from the first embodiment and the first modification of the first embodiment.

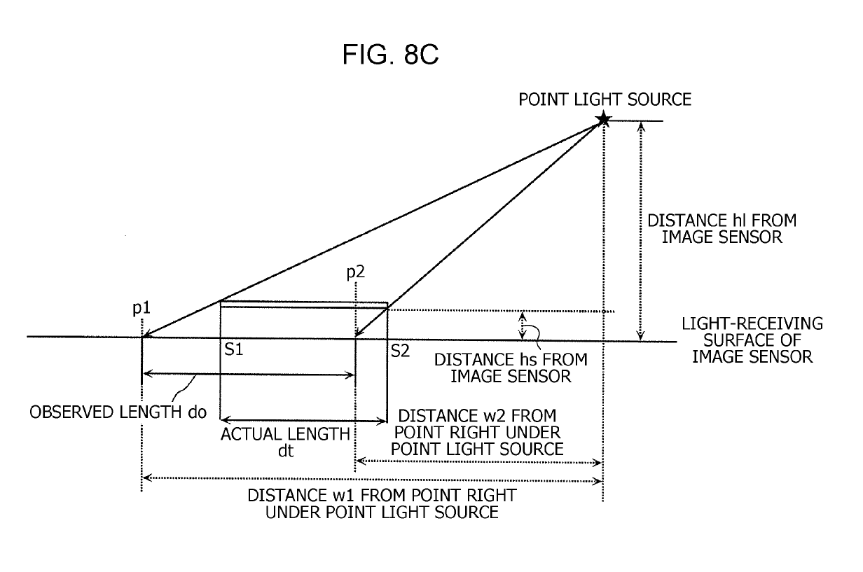

FIG. 9A is a cross-sectional view of a dish-type cell culture container 30A (also simply referred to as the cell culture container 30A) according to the second modification of the first embodiment. FIG. 9B is a cross-sectional view of a flask-type cell culture container 30B (also simply referred to as the cell culture container 30B) according to the second modification of the first embodiment. In FIGS. 9A and 9B, components that are substantially the same as those illustrated in FIGS. 2A and 2B are denoted by the same reference signs, and a detailed description thereof is omitted.

As illustrated in FIGS. 9A and 9B, in the second modification, part of the surface of the lens 128 of an irradiator 320 disposed at the top portion of the cell culture containers 30A and 30B is located in the liquid mixture including the cells 170 and the culture solution 160. That is, part of the surface of the lens 128 is located below the surface 161 of the liquid mixture and above the bottom portion of the container units 100A and 100B.

As described above, in the cell culture containers 30A and 30B according to the second modification, a non-crossing light emission surface (surface of the lens 128) of the irradiator 320 can be placed in the liquid mixture as in the first modification of the first embodiment. Accordingly, refraction of light at the surface 161 of the liquid mixture is successfully avoided, and consequently the shape and dimensions of the cells are successfully derived more easily from the obtained image.

### Third Modification of First Embodiment

A third modification of the first embodiment will be described next. In the first and second modifications of the first embodiment, the non-crossing light emission surface of the irradiator is placed below the surface of the liquid mixture; however, the position of the surface of the liquid mixture changes in accordance with the use state of the cell culture container, especially, an amount of liquid mixture. Accordingly, in the third modification, a mark indicating a reference position of the surface of the liquid mixture is provided on a side portion of the container unit of the cell culture container. If the cell culture container is filled with the liquid mixture up to the height indicated by this mark, the non-crossing light emission surface of the irradiator is placed in the liquid mixture.

The third modification will be described below by focusing on differences from the first embodiment and the first and second modifications of the first embodiment.

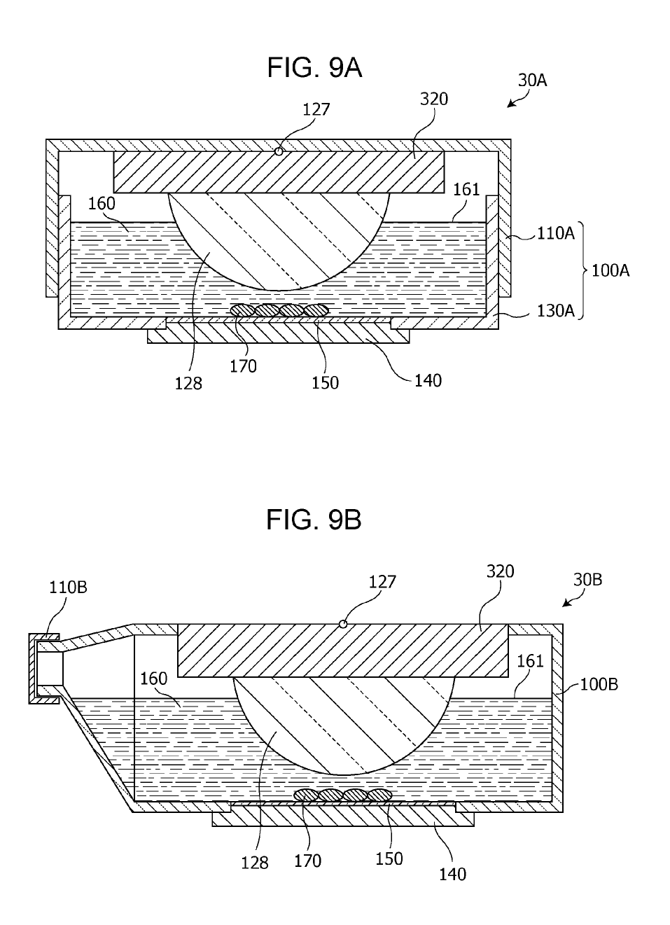

FIG. 10A is a perspective view of a dish-type cell culture container 40A (also simply referred to as the cell culture container 40A) according to the third modification of the first embodiment. FIG. 10B is a perspective view of a flask-type cell culture container 40B (also simply referred to as the cell culture container 40B) according to the third modification of the first embodiment. Note that the lid 110A of the cell culture container 40A is removed from the main body 130A in FIG. 10A. In FIGS. 10A and 10B, components that are substantially the same as those illustrated in FIGS. 1A and 1B are denoted by the same reference signs, and a detailed description thereof is omitted.

As illustrated in FIG. 10A, a mark 131 indicating a reference position of the surface of the liquid mixture (hereinafter, referred to as a reference surface position) is provided on the side portion of the main body 130A (container unit) of the dish-type cell culture container 40A. In addition, as illustrated in FIG. 10B, the mark 131 indicating the reference surface position is provided on the side portion of the container unit 100B of the flask-type cell culture container 40B.

The reference surface position for the liquid mixture indicates a position of the surface corresponding to the lower limit of the amount of liquid mixture necessary for cell culturing. That is, the reference surface position for the liquid mixture is a position of the surface suitable for cell culturing. It is necessary to continuously observe cells in order to culture the cells appropriately. The reference surface position for the liquid mixture is the position of the surface corresponding to the lower limit of the amount of liquid mixture necessary to enable continuous observation of the cells by using the obtained image.

The non-crossing light emission surface of the irradiator 220 is placed below the reference surface position indicated by the mark 131. That is, if the container unit is filled with the liquid mixture up to the reference surface position, the non-crossing light emission surface of the irradiator 220 is located in the liquid mixture.

As described above, in the cell culture containers 40A and 40B according to the third modification, the non-crossing light emission surface of the irradiator 220 can be placed below the reference surface position indicated by the mark 131. Accordingly, if the container unit is filled with the liquid mixture up to the reference surface position, the non-crossing light emission surface is successfully placed in the liquid mixture. Consequently, refraction of the non-crossing light at the surface of the liquid mixture is successfully avoided and the shape and dimensions of the cells are successfully derived more easily from the obtained image.

The mark 131 is a solid line that extends in the horizontal direction in FIGS. 10A and 10B; however, the mark 131 is not limited to this one. For example, the mark 131 may be a dash line or a symbol such as a triangle. In addition, the mark 131 may be printed or may be three-dimensionally formed by changing thickness of the side portion. A single mark 131 is provided on the cell culture containers 40A and 40B in FIGS. 10A and 10B; however, a plurality of marks indicating the reference surface position may be provided.

### Second Embodiment

A second embodiment will be described next. The second embodiment differs from the first modification of the first embodiment in that the side portion or the bottom portion of the container unit has a light-shielding property. The second embodiment will be described below by focusing on differences from the first modification of the first embodiment.

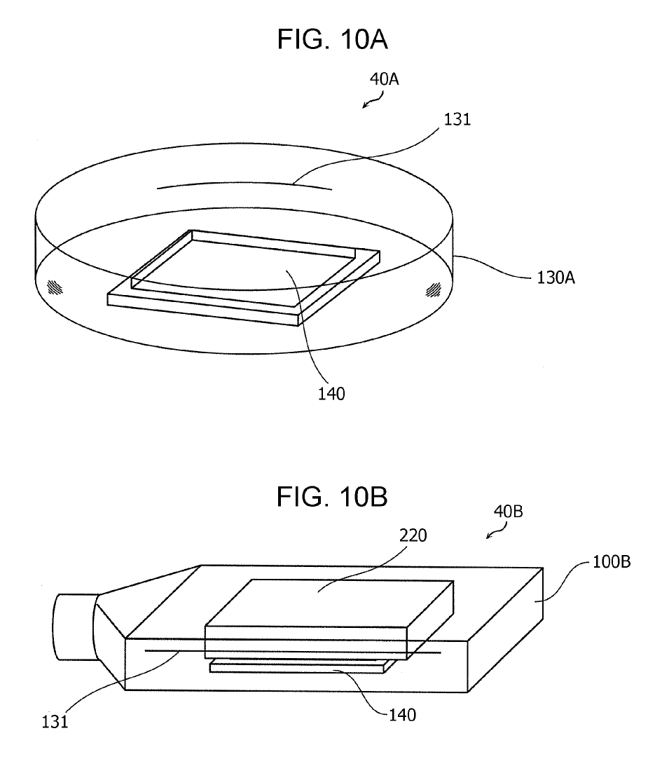

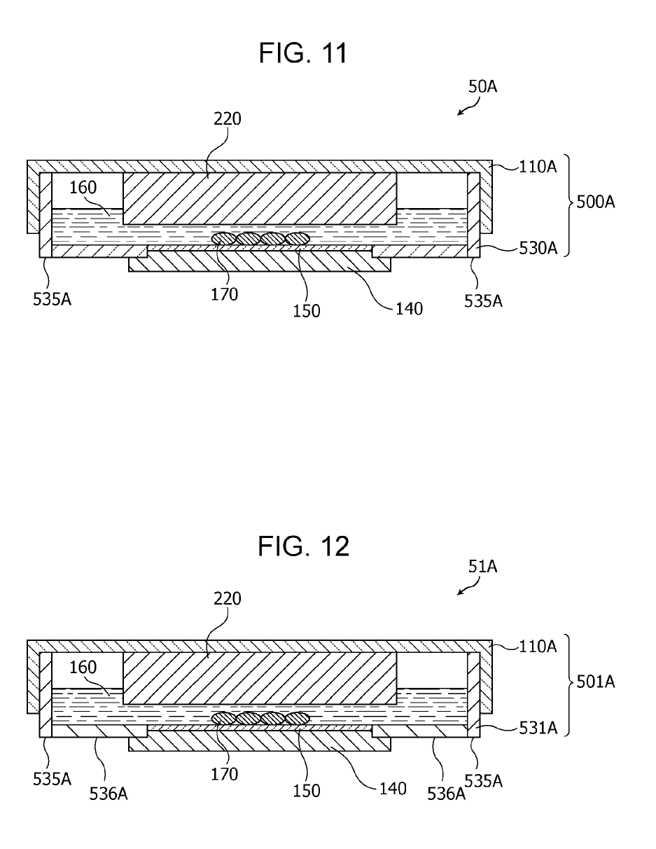

FIG. 11 is a cross-sectional view illustrating an example of a cell culture container 50A according to the second embodiment. FIG. 12 is a cross-sectional view illustrating another example of a cell culture container 51A according to the second embodiment. In FIGS. 11 and 12, components that are substantially the same as those illustrated in FIG. 6A are denoted by the same reference signs, and a detailed description thereof is omitted.

In the example illustrated in FIG. 11, the cell culture container 50A is a dish-type container and includes a container unit 500A, the irradiator 220, and the image sensor 140. The container unit 500A is a container that holds a liquid mixture including the culture solution 160 and the cells 170. The container unit 500A includes the lid 110A and a main body 530A.

The main body 530A is a bottomed cylindrical member that constitutes a bottom portion and a side portion 535A of the container unit 500A. The side portion 535A of the main body 530A has a light-shielding property. Specifically, the side portion 535A of the main body 530A is composed of a material having a light-shielding property and low reflectivity. Alternatively, the side portion 535A of the main body 530A may be covered with a material having a light-shielding property and low reflectivity, for example. The material having a light-shielding property may be, for example, a metal, a black resin, or a carbon fiber.

In the example illustrated in FIG. 12, the cell culture container 51A is a dish-type container and includes a container unit 501A, the irradiator 220, and the image sensor 140. The container unit 501A is a container that holds a liquid mixture including the culture solution 160 and the cells 170. The container unit 501A includes the lid 110A and a main body 531A.

The main body 531A is a bottomed cylindrical member that constitutes a bottom portion and the side portion 535A of the container unit 501A. The side portion 535A of the main body 531A and a part of the bottom portion of the main body 531A where the image sensor 140 is not disposed (part 536A of the bottom portion) have a light-shielding property. Specifically, the side portion 535A and the part 536A of the bottom portion of the main body 531A are composed of a material having a light-shielding property and low reflectivity, for example. Alternatively, the side portion 535A and the part 536A of the bottom portion of the main body 531A may be covered with a material having a light-shielding property and low reflectivity. The material having a light-shielding property may be, for example, a metal, a black resin, or a carbon fiber.

As described above, in the cell culture containers 50A and 51A according to the second embodiment, the side portion of the container unit and/or a part of the bottom portion of the container unit where the image sensor is not disposed can have a light-shielding property. Thus, an amount of ambient light that is incident onto the container unit from outside the container unit is successfully decreased, and consequently an optical image representing the shape and dimensions of the cells more accurately is successfully formed on the light-receiving surface of the image sensor. In particular, when a plurality of cell culture containers are arranged closely adjacent to each other in an incubator, light emitted from the irradiator of one of the plurality of cell culture containers is successfully prevented from reaching the image sensors of the other adjacent cell culture containers, and consequently noise in the image is successfully reduced.

Only the dish-type cell culture containers are illustrated and described in the second embodiment; however, a flask-type cell culture container may have a side portion having a light-shielding property.

### Third Embodiment

A third embodiment will be described next. A cell culture container according to the third embodiment differs from the cell culture containers according to the first and second embodiments in that the cell culture container according to the third embodiment includes connectors that allow an image sensor and an irradiator to communicate with each other. The third embodiment will be described below by focusing on differences from the first and second embodiments.

### Structure of Cell Culture Container

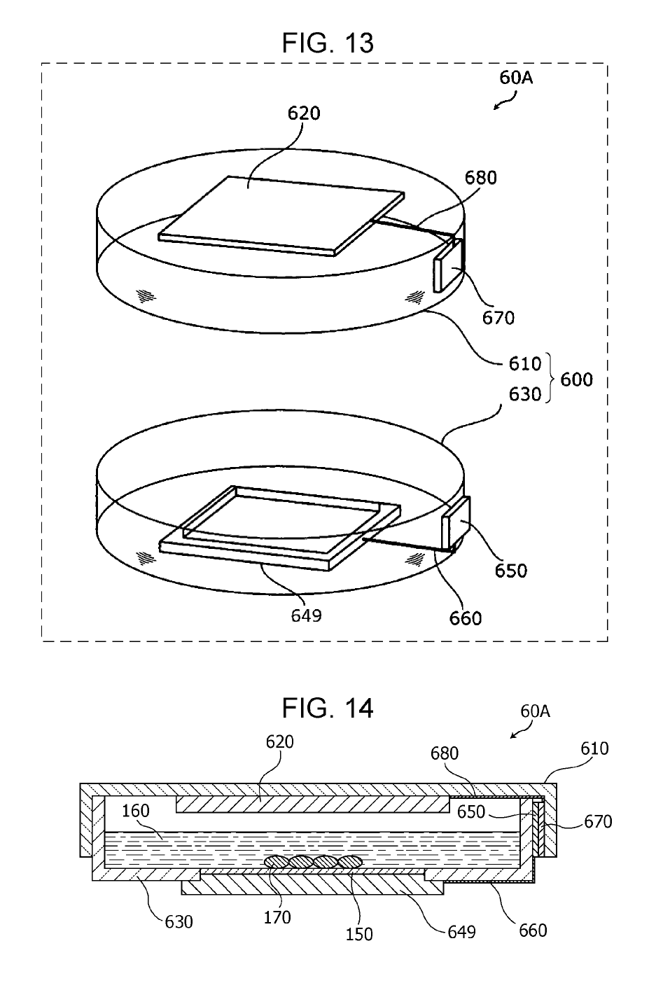

FIG. 13 is a perspective view of a dish-type cell culture container 60A (also simply referred to as the cell culture container 60A) according to the third embodiment. FIG. 13 illustrates a state where a lid 610 is removed from a main body 630.

The cell culture container 60A includes a container unit 600 including the lid 610 and the main body 630, an irradiator 620, and a substrate 649 including an image sensor and a power supply. The cell culture container 60A further includes connectors 650 and 670 and connection lines 660 and 680.

The substrate 649 includes an image sensor and a power supply and is disposed at the bottom portion of the main body 630. The image sensor obtains an optical image of cells formed on the light-receiving surface thereof as a result of irradiation of the cells with non-crossing light.

The irradiator 620 is disposed on the lid 610 and emits non-crossing light. Specifically, the irradiator 620 is fixed to an inner surface of the lid 610 and irradiates the cells held in the container unit 600 with non-crossing light from above. Since the specific configuration of the irradiator 620 is substantially the same as that of the irradiator 120 according to the first embodiment, a description thereof is omitted.

The connector 650 is disposed on an outer side portion of the main body 630. The connector 650 is connected to the substrate 649 via the connection line 660.

The connector 670 is disposed on an inner side portion of the lid 610. The connector 670 is connected to the irradiator 620 via the connection line 680.

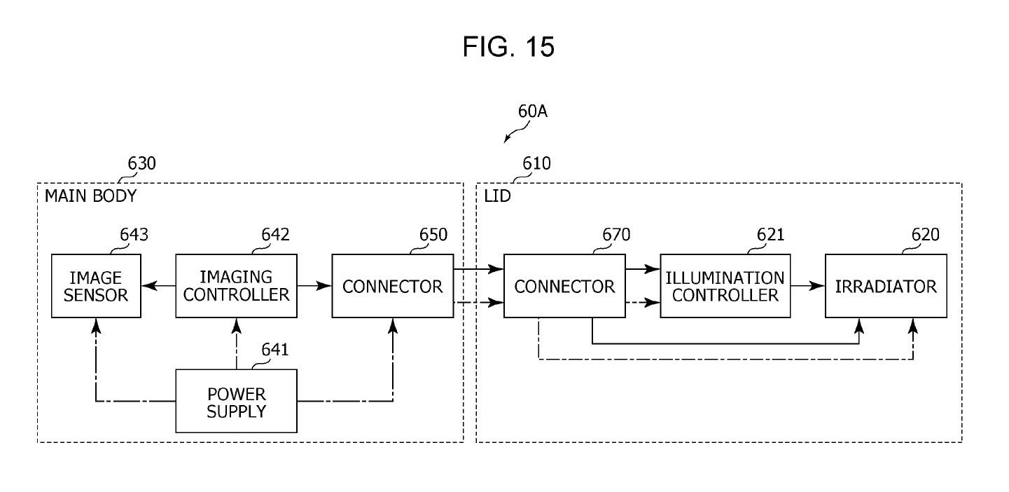

FIG. 14 is a cross-sectional view of the cell culture container 60A when the main body 630 is covered with the lid 610 in the third embodiment.

As illustrated in FIG. 14, when the lid 610 is fitted to the main body 630, the connector 650 disposed on the outer side portion of the main body 630 comes into contact with and is connected to the connector 670 disposed on the inner side portion of the lid 610. Consequently, power is supplied from the substrate 649 including the power supply to the irradiator 620. Further, the irradiator 620 and the substrate 649 including the image sensor communicate with each other.

### Functional Configuration of Cell Culture Container

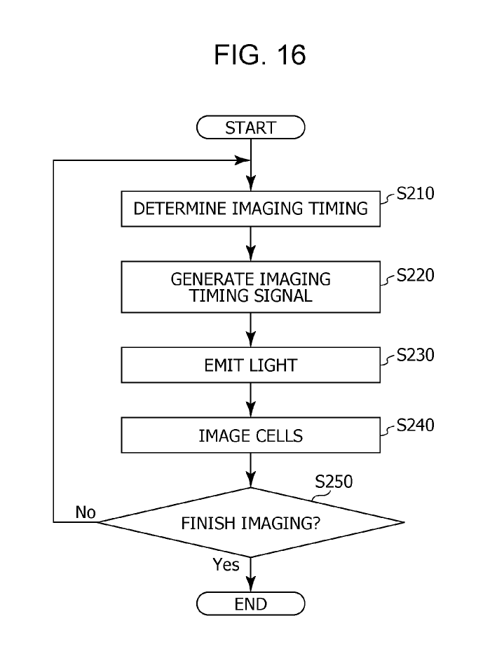

FIG. 15 is a block diagram illustrating a functional configuration of the cell culture container 60A according to the third embodiment. In this block diagram, a solid-line arrow denotes a communication line, and a dot-dash-line arrow denotes a power supply line.

The main body 630 includes an image sensor 643, an imaging controller 642, the connector 650, and a power supply 641. The lid 610 includes the connector 670, an illumination controller 621, and the irradiator 620.

The image sensor 643 includes a plurality of pixels arranged in a matrix. Each of the plurality of pixels outputs an electric signal based on the strength of received light. In this way, the image sensor 643 obtains an image.

The imaging controller 642 controls imaging performed by the image sensor 643. The imaging controller 642 further sends, to the illumination controller 621, a control signal for controlling a timing at which the irradiator 620 emits non-crossing light.

The connector 650 is connected to the connector 670 when the main body 630 is closed by the lid 610. Consequently, the power supply line and the communication line included in the connection line 660 of the main body 630 are connected to the power supply line and the communication line included in the connection line 680 of the lid 610, respectively.

The illumination controller 621 controls emission of non-crossing light from the irradiator 620 in accordance with a control signal supplied from the imaging controller 642.

The irradiator 620 emits non-crossing light used for imaging.

### Operation in Cell Culture Container

An operation performed in the cell culture container 60A configured in the above-described manner will be described next.

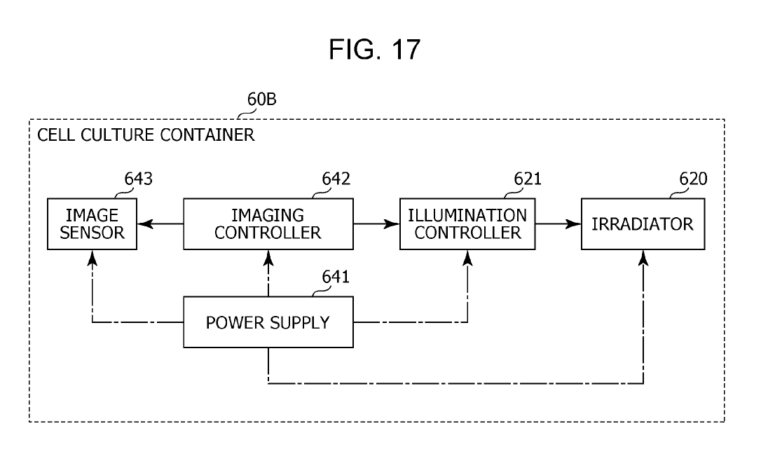

FIG. 16 is a flowchart illustrating an example of the operation performed in the cell culture container 60A according to the third embodiment. First, the imaging controller 642 determines the start of imaging (S210). For example, the imaging controller 642 determines the start of imaging at regular intervals in accordance with an input from a timer (not illustrated). Alternatively, for example, the imaging controller 642 may determine the start of imaging in accordance with an input from an external apparatus (e.g., a user input device).

The imaging controller 642 generates an imaging timing signal for controlling the imaging timing (S220). Then, the imaging controller 642 sends the imaging timing signal generated in step S220 to the illumination controller 621 via the connector 650 of the main body 630 and the connector 670 of the lid 610.

The illumination controller 621 causes the irradiator 620 to emit non-crossing light in accordance with the imaging timing signal received from the imaging controller 642 (S230). That is, the irradiator 620 irradiates cells with non-crossing light in accordance with the imaging timing signal.

On the other hand, the imaging controller 642 causes the image sensor 643 to perform imaging in synchronization with irradiation of the cells with the non-crossing light performed by the irradiator 620 (S240). Consequently, the image sensor 643 obtains an image. The obtained image is stored on a memory (not illustrated), for example. Alternatively, the obtained image may be sent to an external apparatus and stored or displayed on the external apparatus.

Subsequently, the imaging controller 642 determines whether to finish imaging (S250). For example, the imaging controller 642 determines to finish imaging when the imaging has been finished a predetermined number of times. Alternatively, the imaging controller 642 may determine to finish imaging when a period from the start of imaging has exceeded a predetermined period. If it is not determined that imaging is to be finished (No in S250), the process returns to step S210. On the other hand, if it is determined that imaging is to be finished (Yes in S250), the process ends.

In this way, non-crossing light is emitted when the image sensor 643 obtains an image.

The illumination controller 621 may cause the irradiator 620 to stop emitting non-crossing light during a period from step S240 to step S250. In addition, when it is determined in step S250 that imaging is to be finished, the illumination controller 621 may cause the irradiator 620 to stop emitting non-crossing light.

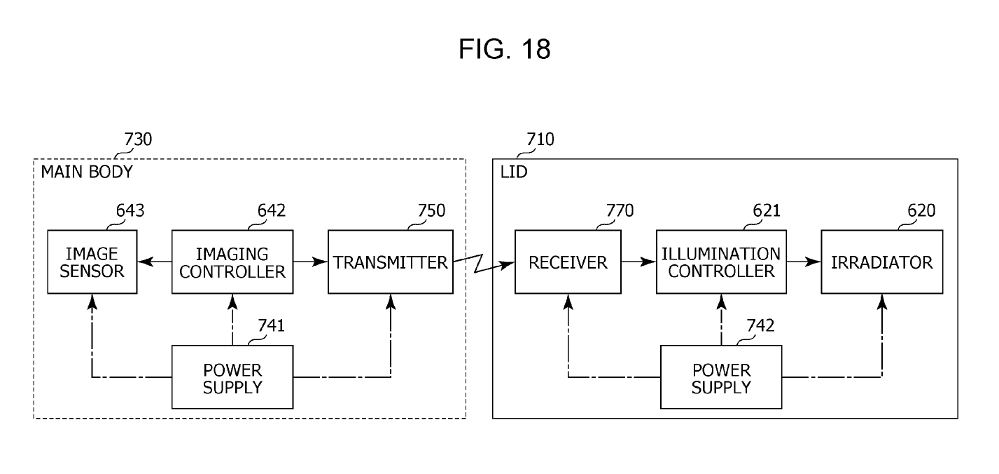

In the third embodiment, the example of the dish-type cell culture container 60A has been described. In the case of a flask-type cell culture container, since the bottom portion where the image sensor is disposed and the top portion where the irradiator is disposed are integrally formed with the side portion interposed therebetween, the connectors are not needed. For example, as illustrated in FIG. 17, the imaging controller 642 and the illumination controller 621 are directly connected to each other without the connectors interposed therebetween in a flask-type cell culture container 60B. In addition, the power supply 641 directly supplies power to the image sensor 643, the imaging controller 642, the illumination controller 621, and the irradiator 620 without the connecters interposed therebetween.

### Advantageous Effects

As described above, since the irradiator 620 and the image sensor 643 can be connected to each other via the connectors 650 and 670 in the cell culture container 60A according to the third embodiment, emission of non-crossing light by the irradiator 620 and imaging by the image sensor 643 are successfully performed in synchronization with each other. Since non-crossing light can be emitted efficiently only during imaging, the energy consumption and the load imposed on the cells by irradiation with the light are successfully reduced.

### First Modification of Third Embodiment

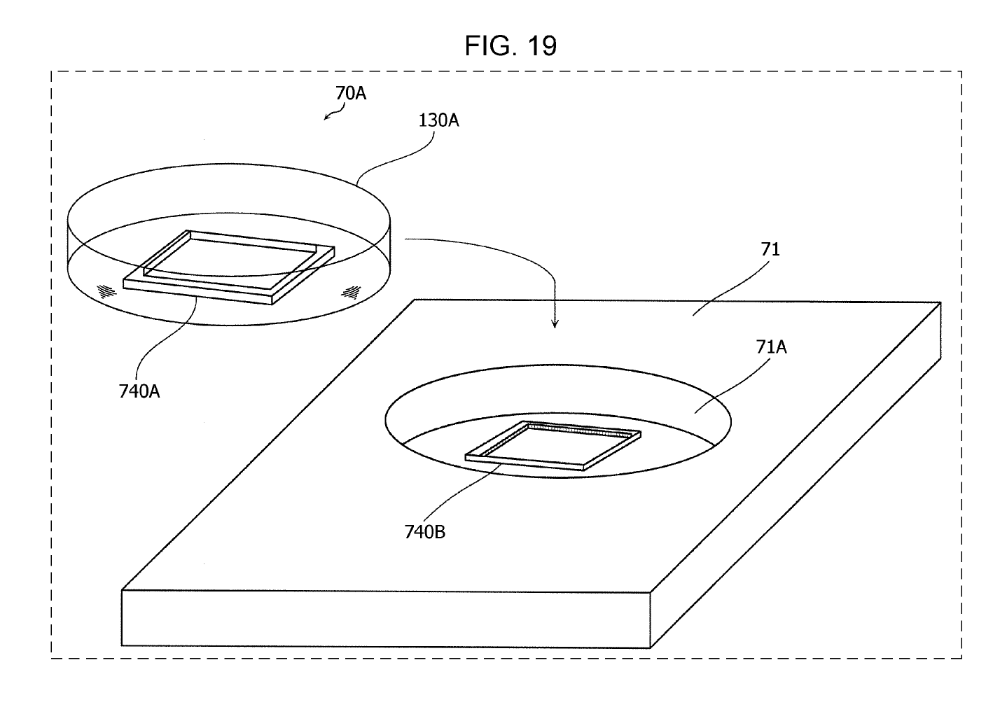

A first modification of the third embodiment will be described next. In the third embodiment described above, the signal is communicated and power is supplied using the connection lines 660 and 680 via the connectors 650 and 670 between the substrate 649 including the image sensor 643 and the power supply 641 disposed on the main body 630 and the irradiator 620 disposed on the lid 610. In the first modification of the third embodiment, a main body 730 includes a transmitter 750 and a lid 710 includes a receiver 770 as illustrated in FIG. 18, and the imaging timing signal is wirelessly transmitted from the imaging controller 642 to the illumination controller 621. In this case, the main body 730 and the lid 710 include power supplies 741 and 742, respectively, as illustrated in FIG. 18. Note that the main body 730 and the lid 710 need not include respective power supplies. For example, the main body 730 may include a power supply, and power may be wirelessly supplied to the lid 710 from the power supply.

### Fourth Embodiment

A fourth embodiment will be described next. In the third embodiment described above, the substrate 649 including an electronic circuit that implements a power supply function and a control function in addition to the image sensor 643 is disposed at the bottom portion of the cell culture container 60A or 60B. In contrast, in the fourth embodiment, a substrate including a power supply circuit and a control circuit is included in a tray on which a cell culture container is mounted. The fourth embodiment will be described below by focusing on differences from the third embodiment.

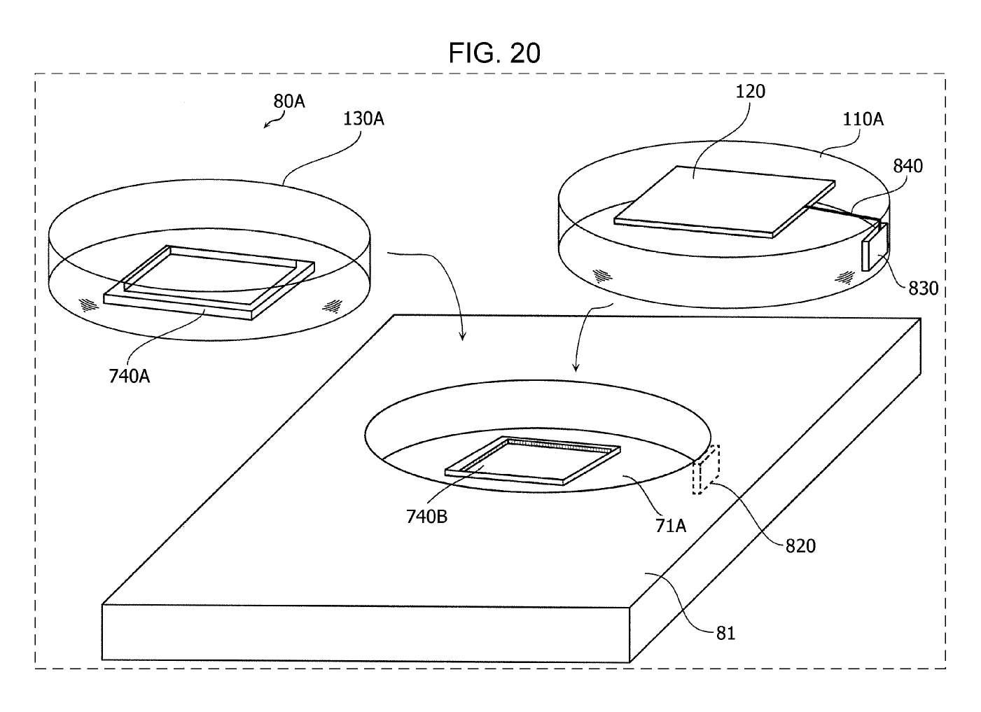

FIG. 19 is a perspective view of a dish-type cell culture container 70A (also simply referred to as the cell culture container 70A) and a tray 71 according to the fourth embodiment. Note that FIG. 19 omits illustration of the lid of the cell culture container 70A.

The tray 71 has a cavity 71A to which the main body 130A of the cell culture container 70A is fitted. A socket 740B to which an image sensor 740A of the cell culture container 70A is detachably attached is disposed at the bottom portion of the cavity 71A. The tray 71 includes an electronic substrate (not illustrated) including a power supply circuit and a control circuit. The electronic substrate and the socket 740B are electrically connected to each other.

The electronic substrate functions as components such as the imaging controller 642 of the third embodiment. Since an operation performed in the cell culture container 70A and the tray 71 when the image sensor 740A is connected to the socket 740B of the tray 71 is substantially the same as that of the third embodiment, a description thereof is omitted.

As described above, the substrate including the power supply circuit and the control circuit can be included in the tray 71 for the cell culture container 70A according to the fourth embodiment. Thus, the substrate including the power supply circuit and the control circuit can be reused, whereas the image sensor that is in direct contact with the cells in the disposable cell culture container is discarded. In this way, cost of cell culturing by using the cell culture container is reduced greatly.

The tray 71 has the cavity 71A for stably holding the cell culture container 70A; however, this cavity 71A need not be formed. That is, the socket 740B may be disposed on a flat plate.

In addition, the electronic substrate disposed inside the tray 71 may be waterproof and exposed to the surface of the tray 71.

### First Modification of Fourth Embodiment

A first modification of the fourth embodiment will be described next. In the fourth embodiment, the example where the image sensor 740A of the main body 130A is connected to the socket 740B to control imaging has been described. In contrast, in the first modification of the fourth embodiment, the irradiator disposed on the lid of the dish-type cell culture container is connected to the electronic substrate disposed in the tray via connectors. The first modification of the fourth embodiment will be described below by focusing on differences from the fourth embodiment.

### Structure of Cell Culture Container and Tray

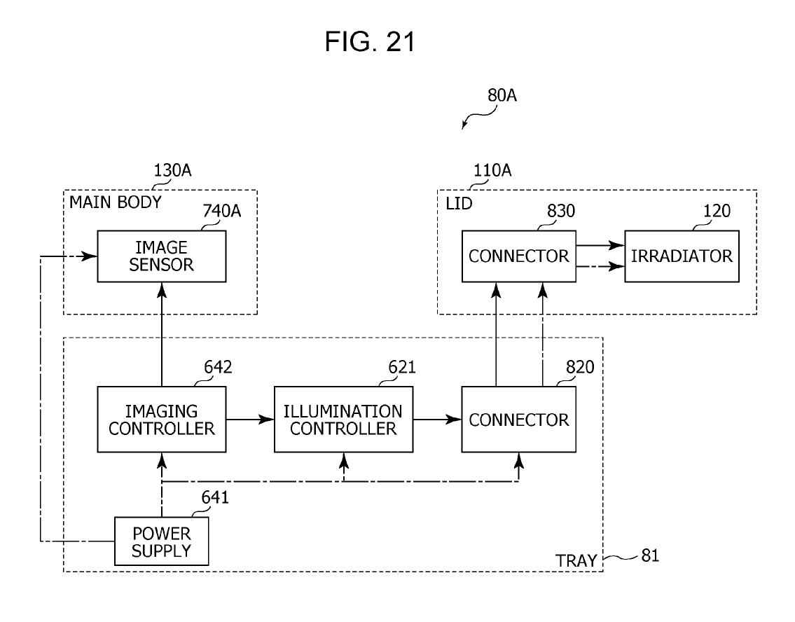

FIG. 20 is a perspective view of a dish-type cell culture container 80A (also simply referred to as the cell culture container 80A) and a tray 81 according to the first modification of the fourth embodiment.

The cell culture container 80A includes the lid 110A, the main body 130A, the image sensor 740A, and the irradiator 120. The image sensor 740A is disposed at the bottom portion of the main body 130A and is connected to the socket 740B of the tray 81 as in the fourth embodiment.

The irradiator 120 is disposed on the lid 110A. In addition, a connector 830 is disposed on the side portion of the lid 110A. The irradiator 120 is connected to the connector 830 via a connection line 840.

The tray 81 has the cavity 71A to which the main body 130A is fitted. The socket 740B to which the image sensor 740A is detachably attached is disposed at the bottom portion of the cavity 71A. Further, a connector 820 connectable to the connector 830 on the lid 110A is disposed on the side portion of the cavity 71A. When the lid 110A is fitted to the main body 130A mounted on the cavity 71A, the connector 820 on the tray 81 is connected to the connector 830 on the lid 110A.

The tray 81 includes an electronic substrate (not illustrated) including a power supply circuit and an electronic circuit. The electronic substrate functions as components such as the imaging controller 642 and the illumination controller 621 of the third embodiment.

### Functional Configuration of Cell Culture Container and Tray

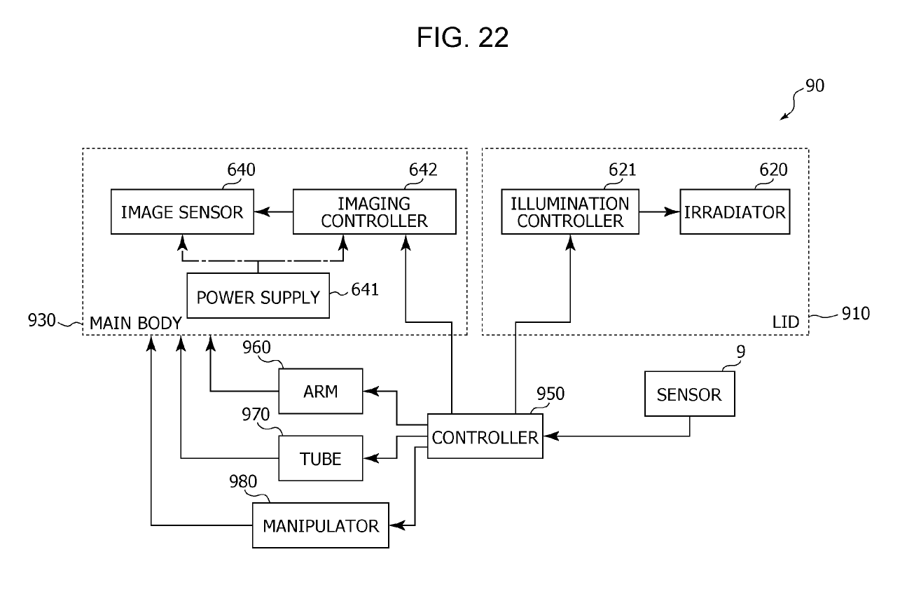

FIG. 21 is a block diagram illustrating a functional configuration of the cell culture container 80A and the tray 81 according to the first modification of the fourth embodiment. In FIG. 21, components substantially the same as those illustrated in FIG. 15 are denoted by the same reference signs, and a detailed description thereof is omitted.

The tray 81 includes the imaging controller 642, the illumination controller 621, the connector 820, and the power supply 641. The lid 110A includes the connector 830 and the irradiator 120. The main body 130A includes the image sensor 740A.

The image sensor 740A can be supplied with power from the power supply 641 and can receive a control signal from the imaging controller 642 when the image sensor 740A is connected to the socket 740B. The image sensor 740A obtains an image in accordance with a control signal received from the imaging controller 642.

The irradiator 120 of the lid 110A can be supplied with power from the power supply 641 and can receive a control signal from the illumination controller 621 when the connector 820 of the tray 81 is connected to the connector 830 of the lid 110A. The irradiator 120 emits non-crossing light in accordance with the control signal received from the illumination controller 621.

### Advantageous Effects

As described above, in the cell culture container 80A and the tray 81 according to the first modification of the fourth embodiment, the substrate including the power supply circuit and the control circuit for the irradiator 120 can be included in the tray 81. Accordingly, the substrate including the power supply circuit and the control circuit can be reused, whereas the image sensor 740A and the irradiator 120 of the disposable cell culture container 80A are discarded. In this way, cost of culturing the cells by using the cell culture container 80A is reduced greatly.

FIG. 19 of the fourth embodiment and FIG. 20 of the first modification of the fourth embodiment illustrate the dish-type cell culture container as an example of the cell culture container; however, the cell culture container may be a flask-type cell culture container. In the case of the flask-type cell culture container, since the bottom portion and the top portion of the container unit are integrally formed with the side portion interposed therebetween, the connectors that connect the irradiator disposed at the top portion and the tray to each other need not be disposed on the side surfaces. The connector may be formed integrally with the socket or may be disposed adjacent to the socket, for example.

### Fifth Embodiment

A fifth embodiment will be described next. A cell culture system according to the fifth embodiment includes a tube 970 used to fill a cell culture container with a culture solution when the cell culture container is used.

### Structure of Cell Culture System

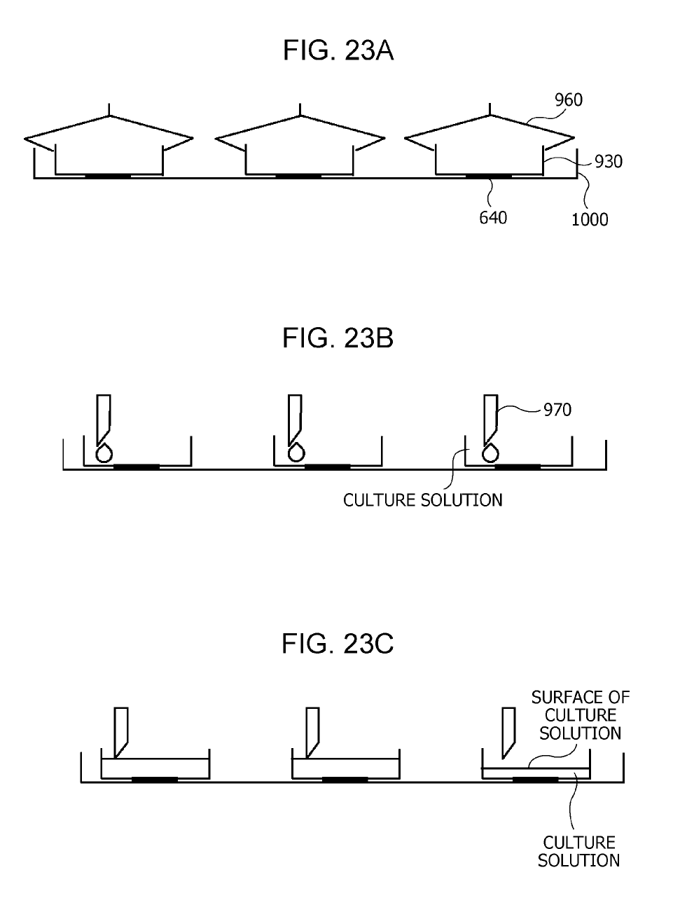

FIG. 22 is a functional block diagram of a cell culture system 90 according to the fifth embodiment. The cell culture system 90 illustrated in FIG. 22 includes a main body 930, a lid 910, a controller 950, an arm 960, the tube 970, and a manipulator 980.

Since the main body 930 and the lid 910 illustrated in FIG. 22 are similar to those illustrated in FIG. 15 except that the connectors 650 and 670 illustrated in FIG. 15 of the third embodiment are removed and a sensor 9 is additionally included, a description will be given by using the same reference signs for components substantially the same as those illustrated in FIG. 15.

The main body 930 includes an image sensor 640, the power supply 641, and the imaging controller 642. The image sensor 640 obtains an image. The imaging controller 642 controls imaging performed by the image sensor 640. The image sensor 640 and the imaging controller 642 are electrically connected to the power supply 641.

The lid 910 includes the irradiator 620 and the illumination controller 621. The irradiator 620 emits parallel light as non-crossing light. The illumination controller 621 controls emission of non-crossing light performed by the irradiator 620.

### Structure of Sensor 9

The sensor 9 detects that the lower surface of the irradiator 620 is in contact with a culture solution.

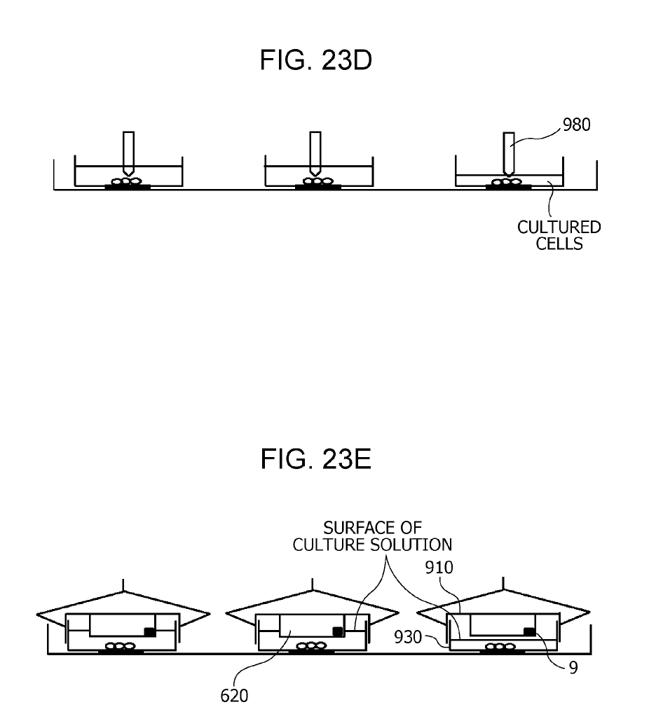

For example, as illustrated in FIG. 23E, the sensor 9 is located inside the main body 930 when the lid 910 is fitted to the main body 930, and the sensor 9 detects that the lower surface of the irradiator 620 is in contact with the culture solution.

Alternatively, the sensor 9 may be fixed to the inner side surface of the main body 930.

A specific example of the sensor 9 may be a moisture sensor placed at the same height as the lower surface of the irradiator 620, that is, the light emission surface. When the moisture sensor detects moisture, it is determined that the lower surface of the irradiator 620 is in contact with the culture solution. In this example, the lid 910 may include the sensor 9.

Another example of the sensor 9 is a set of a camera and a detection circuit. The camera captures an image of the side surface of the main body 930. For example, the camera is disposed on the inner side surface or outer side surface of the main body 930.

The detection circuit detects the height of the surface of the culture solution by using the image captured by the camera to determine whether the lower surface of the irradiator 620 is in contact with the culture solution. For example, the sensor 9 determines the surface of the culture solution by using the image. The sensor 9 then determines whether the surface of the culture solution is above the lower surface of the irradiator 620 depending on whether the detected height of the surface is higher than or equal to a predetermined height.

For example, an image of the surface at the predetermined height is stored on a memory, and pattern matching is performed to compare the captured image with the stored image. In this way, it is determined whether the surface of the culture solution is above the lower surface of the irradiator 620. When the surface in the captured image is above the surface at the predetermined height, the sensor 9 determines that the lower surface of the irradiator 620 is in contact with the culture solution.

The arm 960 is an arm that is used to manipulate the main body 930 and the lid 910.

The arm 960 holds the main body 930 and the lid 910 to move them to a certain position and place them at the certain position.

The tube 970 is used to add the culture solution to the main body 930. The tube 970 is, for example, a conduit that transfers the culture solution from a container (not illustrated) storing the culture solution to the main body 930. When the cell culture system 90 includes a plurality of main bodies 930, the cell culture system 90 may include the same number of tubes 970 as the number of main bodies 930.

The manipulator 980 holds and moves cells. An example of the manipulator 980 is a set of a tube and a pump. The pump is disposed at one of openings of the tube. Cells are sucked at the other opening of the tube and are held in the tube. Then, the cells in the tube are ejected by the pump and are placed in the main body 930.

The controller 950 controls an operation of the cell culture system 90. A predetermined program is stored on a memory in advance, and the controller 950 executes the program to control placement of the main body 930 by the arm 960, addition of the culture solution by using the tube 970, placement of to-be-cultured cells by using the manipulator 980, and placement of the lid 910 by the arm 960.

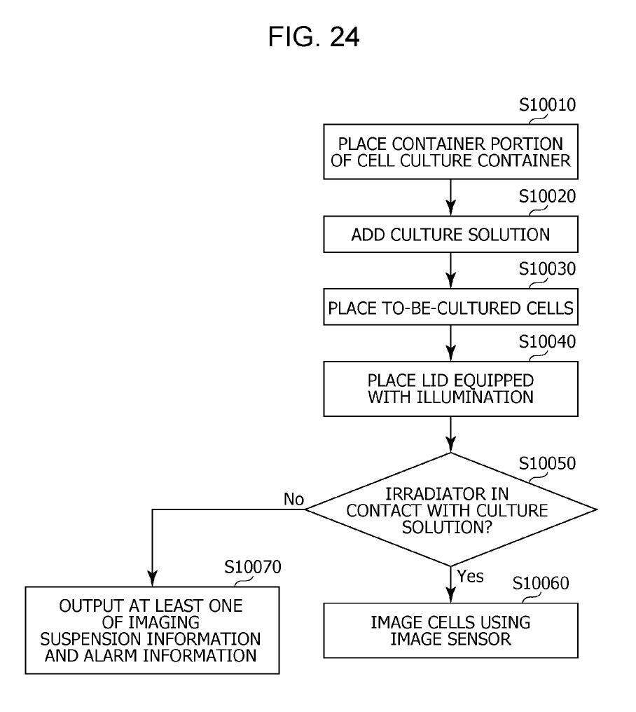

FIGS. 23A to 23E are diagrams schematically illustrating the operation performed by the cell culture system 90. FIG. 24 is a flowchart illustrating the operation performed by the cell culture system 90. A plurality of main bodies 930 are disposed on a tray 1000 illustrated in FIGS. 23A to 23E.

The tray 1000 on which the plurality of main bodies 930 are disposed is merely an example, and a single main body 930 may be placed on the tray 1000.

For example, the tray 1000 is placed in an incubator including a temperature adjuster. The tray 1000 illustrated in FIGS. 23A to 23E may be the tray 1000 placed in an incubator or the tray 1000 removed from the incubator.

### Step S10010

As illustrated in FIG. 23A, the arm 960 places the main body 930 on the tray 1000. More specifically, the arm 960 holds the main body 930 and places the main body 930 on the tray 1000.

The controller 950 outputs, to the arm 960, an movement instruction for causing the arm 960 to hold and move the main body 930. For example, the movement instruction includes a predetermined initial position at which the main body 930 is to be placed and the current position of the main body 930. The controller 950 outputs movement information stored on a memory as the movement instruction. The movement information may be stored on the memory in advance or in response to a user input.

### Step S10020

As illustrated in FIG. 23B, the culture solution is added to the main body 930 by using the tube 970. FIG. 23C illustrates the state where the culture solution has been added to the main body 930.

More specifically, the culture solution is added to the main body 930 by using the tube 970 from a container storing the culture solution.

The controller 950 outputs, to the tube 970, an addition instruction for adding the culture solution to the main body 930. In response to the addition instruction, the tube 970 adds a predetermined kind of culture solution to the main body 930. At that time, the tube 970 adds a predetermined amount of culture solution to the main body 930.

The addition instruction may include information regarding the kind of culture solution and information regarding an amount of culture solution. For example, when the addition instruction includes information regarding the kind of culture solution, the tube 970 sucks the kind of culture solution indicated by the addition instruction from a container storing that kind of culture solution and adds the culture solution to the main body 930.

When a plurality of main bodies 930 are disposed on the tray 1000, the cell culture system 90 may include the tube 970 for each of the plurality of main bodies 930. In addition, the controller 950 may output the addition instruction for each of the plurality of main bodies 930. Alternatively, the controller 950 may output one addition instruction as the instructions for the plurality of main bodies 930.

The controller 950 outputs addition information stored on a memory as the addition instruction. The addition information may be stored on the memory in advance or in response to a user input.

### Step S10030

As illustrated in FIG. 23D, the manipulator 980 places cells in the main body 930. More specifically, the manipulator 980 places cells on the image sensor 640. Consequently, the cells and the culture solution are held in the main body 930. Hereinafter, the culture solution including the cells is also referred to as a liquid mixture.

### Step S10040

The lid 910 including the irradiator 620 is placed on the main body 930. For example, as illustrated in FIG. 23E, the lid 910 is placed on the main body 930 by the arm 960. Steps S10010 to S10040 are preparation for culturing.

### Step S10050

The sensor 9 detects whether the lower surface of the irradiator 620 is located below the surface of the culture solution.

The state where the lower surface of the irradiator 620 is not located below the surface of the culture solution indicates that the predetermined amount of culture solution has not been added to the main body 930. In addition, the state where the lower surface of the irradiator 620 is located below the surface of the culture solution indicates the state where the culture solution is directly irradiated with light from the irradiator 620.

If the sensor 9 detects that the lower surface of the irradiator 620 is located below the surface of the culture solution, the process proceeds to step S10060 (the case of the main bodies 930 located on the left side and at the center in FIG. 23E, for example). The controller 950 sends information indicating that the cell culture system 90 is “ready for imaging” to the imaging controller 642. If the sensor 9 detects that the lower surface of the irradiator 620 is not located below the surface of the culture solution, the process proceeds to step S10070 (the case of the main body 930 located on the right side in FIG. 23E, for example).

### Step S10060

The image sensor 640 images the cells. In other words, the imaging controller 642 causes the image sensor 640 to image the cells.

### Step S10070

The controller 950 outputs imaging suspension information for preventing the image sensor 640 from imaging the cells. For example, the controller 950 outputs the imaging suspension information to the imaging controller 642.

The controller 950 outputs an alarm. The alarm may include information indicating that the lower surface of the irradiator 620 is not located below the surface of the culture solution. The lower surface of the irradiator 620 not being located below the surface of the culture solution equates to the main body 930 not storing the predetermined amount of culture solution. The alarm may also include information indicating that the amount of culture solution is to be increased.

It is sufficient that at least one of the imaging suspension information and the alarm is output.

For example, the controller 950 causes a speaker to output an alarming sound or a sound indicating an abnormality as the alarm. Alternatively, the controller 950 causes a display to display a screen indicating an abnormality. The speaker or the display may be included in the cell culture system 90 or may be an external device of the cell culture system 90.

In the flowchart illustrated in FIG. 24, step S10030 is performed subsequent to step S10020; however, step S10020 may be performed subsequent to step S10030.

In the flowchart illustrated in FIG. 24, step S10050 is performed subsequent to step S10040; however, step S10050 may be performed between step S10020 and step S10030. In this case, if it is determined that the lower surface of the irradiator 620 is not located below the surface of the culture solution, the process proceeds to step S10070. If it is determined that the lower surface of the irradiator 620 is located below the surface of the culture solution, the process proceeds to steps S10030, S10040, and S10060 sequentially.

In addition, step S10050 may be performed between step S10030 and step S10040. In this case, if it is determined that the lower surface of the irradiator 620 is not located below the surface of the culture solution, the process proceeds to step S10070. If it is determined that the lower surface of the irradiator 620 is located below the surface of the culture solution, the process proceeds to steps S10040 and step S10060 sequentially.

In the case where the flow of steps S10010 to S10040 is performed for the tray 1000 placed outside the incubator, the tray 1000 may be moved to inside the incubator by the arm 960 subsequent to step S10040 or if Yes in step S10050. If No in step S10050, the controller 950 does not output, to the arm 960, an instruction to move the tray 1000 to inside the incubator.

It can be determined whether the cell culture system 90 is ready for imaging by determining whether the lower surface of the irradiator 620 is located below the surface of the culture solution immediately before the image sensor 640 performs imaging. That is, when the image sensor 640 performs imaging a plurality of times, step S10050 is repeatedly performed before each imaging operation. In this way, it can be determined whether the current environment is suitable for imaging even if the height of the surface of the culture solution changes during imaging (culturing). For example, if the lower surface of the irradiator 620 is located above the surface of the culture solution, the controller 950 may determine that the current environment is not suitable for imaging and may output an alarm instead of imaging the cells.

### First Modification of Fifth Embodiment

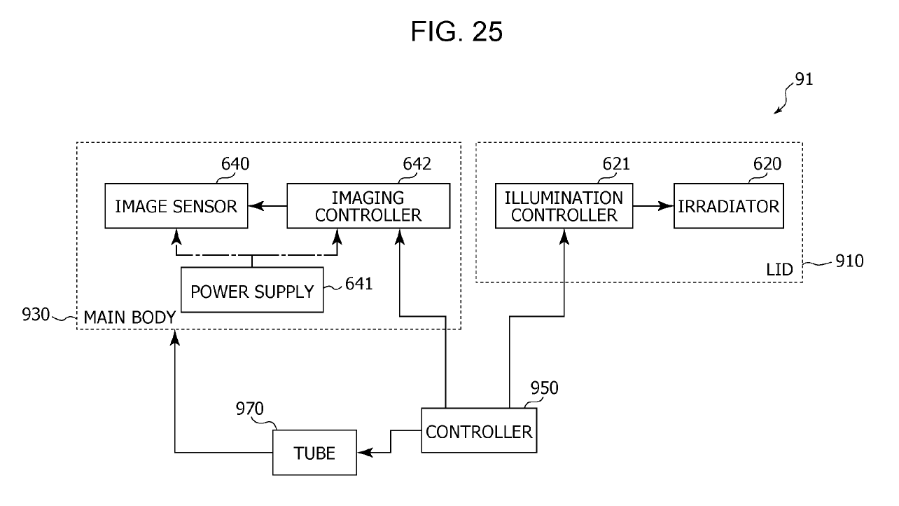

FIG. 25 is a functional block diagram of a cell culture system 91 according to a first modification of the fifth embodiment. The cell culture system 91 according to the first modification of the fifth embodiment includes the main body 930, the lid 910, the controller 950, and the tube 970. That is, unlike the cell culture system 90 according to the fifth embodiment, the cell culture system 91 does not include the arm 960, the manipulator 980, and the sensor 9.

The tube 970 of the cell culture system 91 is similar to the tube 970 of the cell culture system 90. A predetermined amount of culture solution is added to the main body 930 by using the tube 970 (corresponding to step S10020 in FIG. 24). As in the fifth embodiment, the controller 950 controls the tube 970. An example of the predetermined amount of culture solution is an amount of culture solution with which the surface of the culture solution is located at a height higher than or equal to the mark 131 of the third modification of the first embodiment or an amount of culture solution with which the main body 930 becomes full. The image sensor 640 images cells placed in the main body 930 (corresponding to step S10060 in FIG. 24).

### Second Modification of Fifth Embodiment

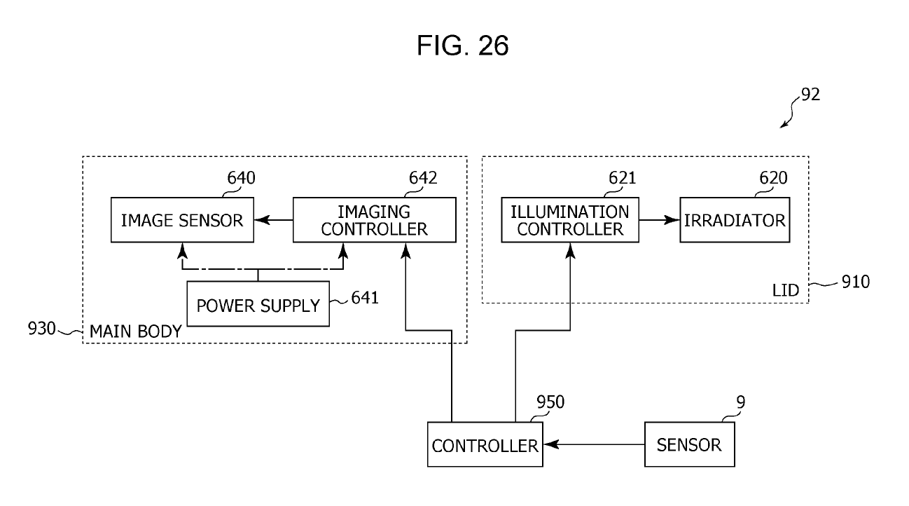

FIG. 26 is a functional block diagram of a cell culture system 92 according to a second modification of the fifth embodiment. The cell culture system 92 includes the main body 930, the lid 910, the controller 950, and the sensor 9.

That is, unlike the cell culture system 90 according to the fifth embodiment, the cell culture system 92 does not include the arm 960, the tube 970, and the manipulator 980.

The sensor 9 of the cell culture system 92 is similar to the sensor 9 of the cell culture system 90. The cell culture system 92 performs steps S10050 to S10070 illustrated in FIG. 24.

### Step S10050

The sensor 9 detects whether the lower surface of the irradiator 620 is located below the surface of the culture solution.

If the sensor 9 detects that the lower surface of the irradiator 620 is not located below the surface of the culture solution, the process proceeds to step S10070 illustrated in FIG. 24.

### Step S10060

The image sensor 640 images cells. In other words, the imaging controller 642 causes the image sensor 640 to image cells.

### Step S10070

The controller 950 outputs imaging suspension information to prevent the image sensor 640 from imaging the cells. For example, the controller 950 outputs the imaging suspension information to the imaging controller 642.

The controller 950 outputs an alarm. It is sufficient that at least one of the imaging suspension information and the alarm information is output.

### Other Embodiments

While the cell culture container according to one or a plurality of aspects of the present disclosure has been described above on the basis of the embodiments, the present disclosure is not limited to these embodiments. Various modifications of the embodiments conceived by a person skilled in the art and embodiments obtained by combining elements of different embodiments with each other may also be within the scope of the one or plurality of aspects of the present disclosure as long as such modifications and embodiments do not depart from the essence of the present disclosure.

For example, in the embodiments described above, the irradiator is disposed on the inner surface of the dish-type cell culture container and on the outer surface of the flask-type cell culture container; however, the configuration is not limited to these ones. The irradiator may be disposed on the outer surface of the dish-type cell culture container and the inner surface of the flask-type cell culture container.

The dish-type and flask-type cell culture containers are described in the embodiments above; however, the shape of the cell culture container is not limited to these ones. The cell culture container may have any given shape suitable for cell culturing.

A single cell culture container is described in the embodiments above; however, a plurality of cell culture containers may be joined together. In this case, a plurality of lids may be provided separately for the plurality of cell culture containers or may be joined together. In addition, the plurality of lids may be formed integrally with a tray cover that covers the tray.

The aspects of the present disclosure are usable as cell culture containers capable of culturing cells and imaging the cultured cells.

What is claimed is:

1. A cell culture container comprising:
a container that houses therein a liquid mixture including one or more cells and a culture solution;
an irradiator that irradiates the liquid mixture with light; and
an image sensor that receives transmitted light,
wherein the transmitted light is the light that has been emitted from the irradiator and has passed through the liquid mixture,
wherein the light emitted from the irradiator includes a plurality of rays, and the plurality of rays do not cross each other between the irradiator and the image sensor,
wherein the container has a mark on a side portion thereof, the side portion is located between a top portion of the container and a bottom portion of the container, and a surface of the liquid mixture is located between the top portion and the bottom portion, and
wherein the irradiator includes an emission surface from which the light is emitted, and the emission surface is located below the surface of the liquid mixture if the container is filled with the liquid mixture up to a height indicated by the mark.

2. The cell culture container according to claim 1, wherein the irradiator is disposed to protrude from the container toward inside the container.

3. The cell culture container according to claim 1, wherein the plurality of rays are parallel to each other between the irradiator and the image sensor.

4. The cell culture container according to claim 3, wherein the irradiator includes a limiting filter that limits a traveling direction of the light, and
wherein the light emitted from the irradiator is light that has passed through the limiting filter.

5. The cell culture container according to claim 3, wherein the irradiator includes a collimating lens, and
wherein the light emitted from the irradiator is light that has passed through the collimating lens.

6. The cell culture container according to claim 1, wherein the plurality of rays are diffused,
wherein the irradiator includes a pinhole, and
wherein the light emitted from the irradiator is light that has passed through the pinhole.

7. The cell culture container according to claim 1, wherein the side portion has a light-shielding property.

8. The cell culture container according to claim 1, wherein the bottom portion of the container has a region not having the image sensor therein, and the region has a light-shielding property.

9. A cell imaging method comprising:
placing an emission surface of an irradiator below a surface of a liquid mixture held in a container, the liquid mixture including one or more cells and a culture solution, the irradiator being configured to emit, from the emission surface, light including a plurality of rays not crossing each other between the irradiator and an image sensor;
irradiating, by the irradiator, the liquid mixture with the light; and
receiving, by the image sensor, transmitted light,
wherein the transmitted light is the light that has been emitted from the irradiator and has passed through the liquid mixture,
wherein the container is filled with the liquid mixture up to a height indicated by a mark, and
wherein the mark is on a side portion of the container, the side portion is located between a top portion of the container and a bottom portion of the container, and the surface of the liquid mixture is located between the top portion and the bottom portion.

10. A cell culture system comprising:
a container that stores a liquid mixture including one or more cells and a culture solution;
an irradiator that irradiates the liquid mixture with light;
an image sensor that images the cells in the container, the image sensor being disposed at a bottom portion of the container;
a sensor that detects whether a predetermined amount of liquid mixture is stored in the container; and
a controller that controls the image sensor,
wherein in a case where the sensor detects that the predetermined amount of liquid mixture is stored in the container, the controller controls the image sensor so as to image the cells in the container, and
wherein in a case where the sensor does not detect that the predetermined amount of liquid mixture is stored in the container, the controller controls the image sensor so as not to image the cells in the container.

11. The cell culture system according to claim 10, wherein the controller further outputs an alarm indicating that the predetermined amount of liquid mixture is not stored in the container in the case where the sensor does not detect that the predetermined amount of liquid mixture is stored in the container.

12. A cell culture container comprising:
a container that houses therein a liquid mixture including one or more cells and a culture solution;
an irradiator that irradiates the liquid mixture with light including first rays that do not cross each other; and
an image sensor that receives resultant light output from the liquid mixture, no condenser lens being provided between the liquid mixture and the image sensor, the resultant light corresponding to the light,
wherein a mark is on a side of the container, and the side is located between a top of the container and a bottom of the container, and
wherein an emission surface of the irradiator from which the light is emitted is located between the bottom and a level indicated by the mark.

13. The cell culture container according to claim 12,
wherein the first rays are third rays while passing through the liquid mixture,
wherein the resultant light includes second rays, and
wherein, with the container housing the liquid mixture from the bottom to the level, the second rays do not cross each other and the third lays do not cross each other.
